(19) United States
(12) Patent Application Publication
(10) Pub. No.: US 2017/0298552 A1
(43) Pub. Date: Oct. 19, 2017

(54) ELECTROLYZED WATER GENERATING DEVICE, ELECTROLYTE FOR GENERATING ELECTROLYZED WATER, AND ELECTROLYZED WATER FOR DISINFECTION

(71) Applicant: Sharp Kabushiki Kaisha, Sakai City, Osaka (JP)

(72) Inventors: Nobutoshi ARAI, Sakai City (JP); Yasuhiro SAKAMOTO, Sakai City (JP); Nobuhiro HAYASHI, Sakai City (JP)

(73) Assignee: Sharp Kabushiki Kaisha, Sakai City, Osaka (JP)

(21) Appl. No.: 15/511,756

(22) PCT Filed: Jan. 28, 2015
(86) PCT No.: PCT/JP2015/052360

§ 371 Date: Mar. 16, 2017

(30) Foreign Application Priority Data
Sep. 19, 2014 (JP) 2014-191556

(51) Int. Cl. D06F 35/00; A61L 2/26; C25B 1/26; C02F 1/467; C02F 1/461; A01N 59/00; A61L 2/18

(52) U.S. Cl. CPC D06F 35/003; A61L 2/18; A61L 2/26; D06F 35/005; C02F 1/467; C02F 1/46104; A01N 59/00; C25B 1/26; A61L 2202/11; A61L 2202/17; C02F 2201/4611; A61L 2202/26

(57) ABSTRACT

An electrolyzed water generating device of the present invention includes an electrolytic solution supplying unit and an electrolysis unit including an electrolysis electrode pair. The electrolytic solution supplying unit is provided so as to supply an aqueous solution of an electrolyte for generating electrolyzed water to the electrolysis unit. The electrolysis unit is provided so that the aqueous solution of the electrolyte for generating electrolyzed water is electrolyzed using the electrolysis electrode pair to generate an electrolyzed water. The electrolyte for generating electrolyzed water contains an alkali metal chloride and a substance that makes an aqueous solution acidic. The electrolyzed water generating device generates an electrolyzed water having a pH of more than 6.5 and less than 8.0.

### TECHNICAL FIELD

The present invention relates to an electrolyzed water generating device, an electrolyte for generating electrolyzed water, and an electrolyzed water for disinfection.

### BACKGROUND ART

Since an electrolyzed water containing hypochlorous acid produces a disinfecting effect, such an electrolyzed water is used in order to, for example, prevent infectious diseases, maintain the freshness of perishable foods, and deodorize laundry.

There have been known an electrolysis device in which an aqueous solution containing hydrogen chloride is electrolyzed to generate an electrolyzed water having a pH of 6.3 or less, and a method for cleaning clothes using an electrolyzed water generated by electrolyzing an aqueous solution containing hydrogen chloride and having a pH of 6 or less (e.g., refer to PTL 1 and PTL 2).

There has also been known a washing machine that uses an electrolyzed water generated by electrolyzing a saline solution (e.g., refer to PTL 3 to PTL 5).

There has also been known a technique in which hydrochloric acid or acetic acid is added to an electrolyzed water generated by electrolyzing an aqueous solution containing sodium chloride (e.g., refer to PTL 6 and PTL 7).

### CITATION LIST

### Patent Literature

PTL 1: Japanese Unexamined Patent Application Publication No. 2005-138093

PTL 2: Japanese Unexamined Patent Application Publication No. 2007-135758

PTL 3: Japanese Unexamined Patent Application Publication No. 2001-170392

PTL 4: Japanese Unexamined Patent Application Publication No. 2013-102921

PTL 5: Japanese Unexamined Patent Application Publication No. 2013-132342

PTL 6: Japanese Patent No. 3951156

PTL 7: Japanese Unexamined Patent Application Publication No. 2013-102919

### SUMMARY OF INVENTION

### Technical Problem

However, when an acidic electrolyzed water is used, chlorine gas is easily generated from the electrolyzed water and the fading of and damage to objects to be disinfected are easily caused. An electrolyzed water generated by electrolyzing a saline solution is alkaline and thus the disinfecting effect is relatively low. Furthermore, if hydrochloric acid or the like is added to an electrolyzed water, the configuration of the electrolysis device is complicated, which increases the device size.

In view of the foregoing, the present invention provides an electrolyzed water generating device that efficiently generates a highly disinfectant electrolyzed water with which the fading of and damage to objects to be disinfected can be suppressed.

### Solution to Problem

The present invention provides an electrolyzed water generating device including an electrolytic solution supplying unit and an electrolysis unit including an electrolysis electrode pair, wherein the electrolytic solution supplying unit is provided so as to supply an aqueous solution of an electrolyte for generating electrolyzed water to the electrolysis unit, the electrolysis unit is provided so that the aqueous solution of the electrolyte for generating electrolyzed water is electrolyzed using the electrolysis electrode pair to generate an electrolyzed water, the electrolyte for generating electrolyzed water contains an alkali metal chloride and a substance that makes an aqueous solution acidic, and the electrolyzed water generating device generates an electrolyzed water having a pH of more than 6.5 and less than 8.0.

### Advantageous Effects of Invention

According to the present invention, the electrolyzed water generating device includes the electrolytic solution supplying unit provided so as to supply an aqueous solution of an electrolyte for generating electrolyzed water to the electrolysis unit and the electrolysis unit provided so that the aqueous solution of the electrolyte for generating electrolyzed water is electrolyzed using the electrolysis electrode pair to generate an electrolyzed water. Therefore, an electrolyzed water can be produced from the aqueous solution of the electrolyte for generating electrolyzed water.

According to the present invention, the electrolyte for generating electrolyzed water contains an alkali metal chloride. Therefore, an electrolyzed water containing hypochlorous acid, a hypochlorite, and an alkali metal chloride can be produced by the electrolysis unit.

According to the present invention, the electrolyte for generating electrolyzed water contains an alkali metal chloride and a substance that makes an aqueous solution acidic. Therefore, an electrolyzed water having a pH of more than 6.5 and less than 8.0 can be produced. Thus, a substantially neutral electrolyzed water can be generated. Even if the electrolyzed water adheres to the skin, the damage to the skin can be suppressed. When clothes, towels, and the like are disinfected using the generated electrolyzed water, the damage to and fading of a cloth can be suppressed. Furthermore, since the electrolyzed water has a pH of more than 6.5, the generation of chlorine gas can be suppressed.

According to the present invention, an electrolyzed water having a low effective chlorine concentration but a high disinfecting effect can be generated. Therefore, the generation cost of the electrolyzed water can be reduced. Furthermore, a large amount of electrolyzed water can be generated within a short time. This has been demonstrated through the experiment conducted by the present inventors and the like.

According to the present invention, since there is no need to add an acidic substance to the electrolyzed water, the size of the electrolyzed water generating device can be decreased.

### BRIEF DESCRIPTION OF DRAWINGS

FIG. 1 is a schematic sectional view of an electrolyzed water generating device according to an embodiment of the present invention.

FIG. 2 is a schematic configuration diagram of an electrolyzed water generating device according to an embodiment of the present invention.

FIG. 3 is a schematic sectional view of an electrolyzed water generating device according to an embodiment of the present invention.

FIG. 4 is a schematic sectional view of an electrolyzed water generating device according to an embodiment of the present invention.

FIGS. 5(a) to 5(c) are schematic sectional views of stirring units included in an electrolyzed water generating device according to an embodiment of the present invention, and FIG. 5(d) is a schematic sectional view of an air bubble dividing unit included in the stirring unit.

FIG. 6(a) is a schematic vertical sectional view of a stirring unit included in an electrolyzed water generating device according to an embodiment of the present invention, and FIGS. 6(b) to 6(e) are schematic views obtained by projecting stirring units in the vertical direction.

FIGS. 7(a) to 7(d) are schematic sectional views of electrolyzed water generating devices produced in an electrolyzed water generation experiment.

FIGS. 8(a) and 8(b) are graphs showing the measurement results of an electrolyzed water generation experiment 2.

FIG. 9 is a graph showing the results of a disinfection experiment.

FIG. 10 is a graph showing the results of a disinfection experiment.

FIG. 11 is a graph showing the results of a disinfection experiment.

FIGS. 12(a) and 12(b) are flowcharts illustrating washing processes in a washing experiment.

### DESCRIPTION OF EMBODIMENTS

An electrolyzed water generating device of the present invention includes an electrolytic solution supplying unit and an electrolysis unit including an electrolysis electrode pair. The electrolytic solution supplying unit is provided so as to supply an aqueous solution of an electrolyte for generating electrolyzed water to the electrolysis unit. The electrolysis unit is provided so that the aqueous solution of the electrolyte for generating electrolyzed water is electrolyzed using the electrolysis electrode pair to generate an electrolyzed water. The electrolyte for generating electrolyzed water contains an alkali metal chloride and a substance that makes an aqueous solution acidic. The electrolyzed water generating device generates an electrolyzed water having a pH of more than 6.5 and less than 8.0.

In the electrolyzed water generating device of the present invention, an electrolyzed water having an effective chlorine concentration of 10 ppm or more and 100 ppm or less is preferably generated.

In this configuration, a highly disinfectant electrolyzed water with which the fading of objects to be disinfected can be suppressed can be generated. Furthermore, the electrolyzed water can be efficiently generated, and thus a large amount of highly disinfectant electrolyzed water can be generated.

In the electrolyzed water generating device of the present invention, the alkali metal chloride is preferably at least one of sodium chloride and potassium chloride.

When the electrolyte for generating electrolyzed water contains potassium chloride, the cleaning properties of the generated electrolyzed water against oil soils can be improved. Furthermore, the generated electrolyzed water can be sprayed to crops in order to prevent blight, for example.

When the electrolyte for generating electrolyzed water contains sodium chloride, the generation cost of the electrolyzed water can be reduced.

In the electrolyzed water generating device of the present invention, the substance that makes an aqueous solution acidic is preferably hydrogen chloride.

In this configuration, hydrogen chloride can be electrolyzed to generate hypochlorous acid, which increases the effective chlorine concentration of the electrolyzed water.

The electrolyzed water generating device of the present invention preferably further includes a diluting unit, and the diluting unit is configured to dilute the electrolyzed water generated by the electrolysis unit with water.

In this configuration, the electrolyzed water generated by the electrolysis unit can be diluted with water to generate an electrolyzed water having an effective chlorine concentration of 10 ppm or more and 100 ppm or less. Therefore, the amount of an electrolyzed water generated can be increased. Furthermore, the concentration of the electrolyzed water can be easily adjusted by changing the amount of water used for dilution.

The electrolyzed water generating device of the present invention preferably further includes a stirring unit, and the stirring unit is configured to stir the electrolyzed water diluted by the diluting unit.

In this configuration, the concentration unevenness of the electrolyzed water can be suppressed, and the effective chlorine concentration, pH, and the like of the electrolyzed water generated can be stabilized.

The present invention also provides an electrolyte for generating electrolyzed water, the electrolyte containing an alkali metal chloride and a substance that makes an aqueous solution acidic.

When the electrolyte for generating electrolyzed water of the present invention is used, an electrolyzed water having a pH of more than 6.5 and less than 8.0 can be generated. Furthermore, an electrolyzed water having a low effective chlorine concentration but a high disinfecting effect can be generated.

The present invention also provides an electrolyzed water for disinfection generated by electrolyzing an aqueous solution of an electrolyte for generating electrolyzed water, the electrolyte containing an alkali metal chloride and a substance that makes an aqueous solution acidic. The electrolyzed water for disinfection has a pH of more than 6.5 and less than 8.0.

The electrolyzed water for disinfection of the present invention is substantially neutral. Therefore, even if the electrolyzed water adheres to the skin, the damage to the skin can be suppressed. When clothes, towels, and the like are disinfected using the electrolyzed water for disinfection, the damage to and fading of a cloth can be suppressed. Furthermore, since the electrolyzed water has a pH of more than 6.5, the generation of chlorine gas from the electrolyzed water for disinfection can be suppressed. The electrolyzed water for disinfection of the present invention has a low effective chlorine concentration, but a high disinfecting effect. This has been demonstrated through the experiment conducted by the present inventors and the like.

Hereafter, embodiments of the present invention will be described with reference to the attached drawings. The configurations shown in the drawings and the description below are merely examples. The scope of the present invention is not limited to those shown in the drawings and the description below.

### First Embodiment

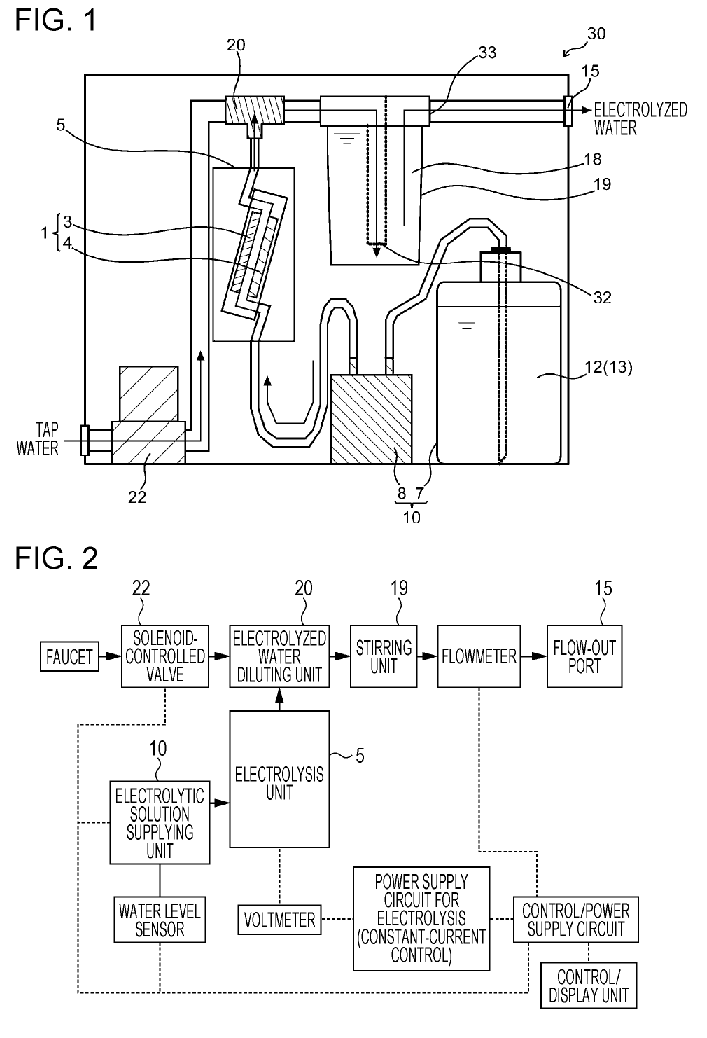

FIG. 1 is a schematic sectional view of an electrolyzed water generating device according to a first embodiment. FIG. 2 is a schematic configuration diagram of the electrolyzed water generating device according to the first embodiment.

An electrolyzed water generating device 30 according to this embodiment includes an electrolytic solution supplying unit 10 and an electrolysis unit 5 including an electrolysis electrode pair 1. The electrolytic solution supplying unit 10 is provided so as to supply an aqueous solution of an electrolyte for generating electrolyzed water to the electrolysis unit 5. The electrolysis unit 5 is provided so that the aqueous solution of the electrolyte for generating electrolyzed water is electrolyzed using the electrolysis electrode pair 1 to generate an electrolyzed water. The electrolyte for generating electrolyzed water contains an alkali metal chloride and a substance that makes an aqueous solution acidic. The electrolyzed water generating device 30 generates an electrolyzed water having a pH of more than 6.5 and less than 8.0.

Hereafter, an electrolyzed water generating device 30 according to this embodiment will be described.

### 1. Electrolyzed Water and Electrolyzed Water Generating Device

The electrolyzed water 18 is an aqueous solution containing a reaction product of electrolysis reaction. The electrolyzed water generating device 30 is a device for producing the electrolyzed water 18.

In this embodiment, the electrolyzed water generating device 30 is configured to generate an electrolyzed water 18 containing hypochlorous acid (HClO), a hypochlorite (e.g., NaClO and KClO), and an alkali metal chloride. The electrolyzed water generating device 30 may be a standalone device or a unit incorporated into another device and used for generating an electrolyzed water 18. For example, in the case of a washing machine, the electrolyzed water generating device 30 may be a unit included in the washing machine and used for generating an electrolyzed water 18.

The electrolyzed water generating device 30 generates an electrolyzed water 18 having a pH of more than 6.5 and less than 8.0, preferably an electrolyzed water 18 having a pH of 7.0 or more and 7.5 or less. This improves the disinfecting effect of the electrolyzed water 18. When the pH of the electrolyzed water 18 is more than 6.5, the fading of and damage to fibers of disinfected clothes and the like can be suppressed. Furthermore, the generation of chlorine gas from the electrolyzed water 18 can be suppressed.

When the pH of the electrolyzed water 18 is less than 8.0, the disinfecting effect of the electrolyzed water can be improved. Thus, an electrolyzed water that produces a sufficiently high disinfecting effect can be generated at a low effective chlorine concentration. The generation cost of the electrolyzed water can also be reduced.

The electrolyzed water generating device 30 can generate an electrolyzed water 18 having an effective chlorine concentration of 10 ppm or more and 100 ppm or less. The electrolyzed water generating device 30 can also generate an electrolyzed water 18 having an effective chlorine concentration of 20 ppm or more and 50 ppm or less. This improves the disinfectant properties while fading of objects to be disinfected is suppressed.

The electrolyzed water 18 generated by the electrolyzed water generating device 30 may contain hypochlorous acid, a hypochlorite (e.g., sodium hypochlorite and potassium hypochlorite), and an alkali metal chloride (e.g., sodium chloride and potassium chloride). When the electrolyzed water 18 contains hypochlorous acid, the electrolyzed water 18 has a high disinfecting effect. When the electrolyzed water 18 contains a hypochlorite, the electrolyzed water 18 has good cleaning properties against organic soils. When the electrolyzed water 18 contains an alkali metal chloride, the electrolyzed water 18 has good cleaning properties against oil soils. Furthermore, the permeability into gaps of fibers and the like is improved, which improves the disinfecting and cleaning properties. The disinfecting effect of the electrolyzed water 18 can also be improved. As described above, when the electrolyzed water 18 contains hypochlorous acid, a hypochlorite, and an alkali metal chloride, the electrolyzed water 18 can produce a high disinfecting effect and a high cleaning effect.

When the electrolyzed water 18 contains hypochlorous acid, a hypochlorite, and an alkali metal chloride, the concentration of the alkali metal chloride is preferably higher than those of the hypochlorous acid and the hypochlorite. In this case, an optimum electrolyzed water for washing is obtained because of the individual characteristics and synergistic effect of the hypochlorous acid, hypochlorite, and alkali metal chloride. Furthermore, the concentration of the hypochlorous acid is preferably higher than that of the hypochlorite. The concentration of the alkali metal chloride is more preferably higher than the total concentration of the hypochlorous acid and the hypochlorite. This order relation can be simply evaluated on the basis of effective chlorine concentration chloride concentration.

Substantially all or 50% or more of alkali metal ions contained in the electrolyzed water 18 may be potassium ions. This increases the cleaning properties of the electrolyzed water 18 against oil soils.

Substantially all or 50% or more of alkali metal ions contained in the electrolyzed water 18 may be sodium ions. This reduces the generation cost of the electrolyzed water 18.

The effective chlorine concentration of the electrolyzed water can be set to 100 ppm or less and preferably 50 ppm or less to suppress fading. An electrolyzed water having higher concentration can be used for articles in which the fading of stainless tools and the like and the damage to fibers are not required to be considered. However, if the concentration is excessively high, the concentration after generation quickly decreases, which makes it difficult to control the concentration or may cause generation of chlorine gas. Therefore, the concentration is preferably 1000 ppm or less and preferably 300 ppm or less. Obviously, the concentration is not limited thereto as long as safety can be secured, for example, disinfection and cleaning are performed in a fully sealed device. The electrolyzed water may be used in any concentration that is suitable for those to be cleaned.

The ratio of hypochlorous acid (HClO) and a hypochlorite (e.g., NaClO and KClO) contained in the electrolyzed water 18 may be 1:9 to 9:1 and is preferably 2:8 to 5:5. This offers a good balance of a disinfecting effect and a bleaching effect of the electrolyzed water 18.

When the ratio of the hypochlorous acid (HClO) is high and the concentration of the hypochlorous acid is high, the electrolyzed water has good disinfectant properties. A faintly acidic disinfecting water having a hypochlorous acid concentration of 90% or more and an apparatus for generating such disinfecting water are commercially available. However, if such disinfecting water is directly used for disinfection and cleaning of, for example, clothes, carpets, floors, and walls, fading and damage to materials such as fibers become severe.

On the other hand, in the electrolyzed water having a high ratio of the hypochlorite, the disinfection treatment time needs to be increased and the concentration of the hypochlorite needs to be increased. If the treatment time is increased or the concentration of the hypochlorite is increased, the fading of and damage to fibers increase.

Therefore, when the electrolyzed water 18 generated by the electrolyzed water generating device 30 according to this embodiment has an optimum ratio of the hypochlorous acid and the hypochlorite, the disinfectant properties can be improved while the fading and the damage to fibers are further suppressed compared with a known disinfecting water and a commercially available bleaching solution.

### 2. Electrolytic Solution Supplying Unit and Electrolyte for Generating Electrolyzed Water

The electrolytic solution supplying unit 10 is provided so as to supply an aqueous solution of the electrolyte 13 for generating electrolyzed water to the electrolysis unit 5. Thus, the aqueous solution of the electrolyte 13 for generating electrolyzed water can be electrolyzed by the electrolysis unit 5. The electrolyte 13 for generating electrolyzed water may be an electrolytic stock solution 12 that can be directly supplied to the electrolysis unit 5, a concentrated electrolytic solution, or a powdery electrolyte.

The electrolyte 13 for generating electrolyzed water contains an alkali metal chloride and a substance that makes an aqueous solution acidic. The alkali metal chloride is preferably sodium chloride or potassium chloride. The electrolyte 13 for generating electrolyzed water may contain both sodium chloride and potassium chloride.

When the electrolyte 13 for generating electrolyzed water contains an alkali metal chloride, the electrolyzed water generated by the electrolyzed water generating device 30 can contain hypochlorous acid and a hypochlorite, which imparts the electrolyzed water 18 to a disinfecting effect. Furthermore, an alkaline substance generated through electrolysis of the alkali metal chloride increases the pH of the electrolyzed water 18 generated by the electrolyzed water generating device 30 to more than 6.5. When the electrolyte 13 for generating electrolyzed water contains an alkali metal chloride, the electrolyzed water 18 can contain the alkali metal chloride.

When the electrolyte 13 for generating electrolyzed water contains sodium chloride, which is less expensive, the production cost of the electrolyzed water can be reduced. When the electrolyte 13 for generating electrolyzed water contains potassium chloride, the electrolyzed water produced contains potassium ions. Thus, the electrolyzed water can be sprayed to crops in order to prevent blight, for example. In this case, potassium ions can be used as fertilizer.

When the electrolyte 13 for generating electrolyzed water contains a substance that makes an aqueous solution acidic, the pH of the electrolyzed water 18 generated by the electrolyzed water generating device 30 can be decreased to less than 8.0. Examples of the “substance that makes an aqueous solution acidic” contained in the electrolyte 13 for generating electrolyzed water include hydrogen chloride (hydrochloric acid), sulfuric acid, nitric acid, acetic acid, citric acid, and hydrogen fluoride (hydrofluoric acid). The substance that makes an aqueous solution acidic is preferably hydrogen chloride. Thus, hypochlorous acid can be produced from chlorine ions contained in hydrogen chloride, which increases the effective chlorine concentration of the electrolyzed water generated. The substance that makes an aqueous solution acidic may be citric acid. Thus, the electrolyte 13 for generating electrolyzed water can be treated as a mixed powder of a solid alkali metal chloride and a solid citric acid. Consequently, the electrolyte 13 for generating electrolyzed water can be easily handled.

The electrolytic solution supplying unit 10 may include an electrolytic solution tank 7 that stores the electrolytic stock solution 12 serving as the electrolyte 13 for generating electrolyzed water and a pump 8 that supplies the electrolytic stock solution 12 to the electrolysis unit 5 as in the electrolyzed water generating device 30 illustrated in FIG. 1. In this case, the electrolytic solution tank 7 can store an electrolytic stock solution 12 in which the concentration of the alkali metal chloride and the concentration of the substance that makes an aqueous solution acidic are optimized for electrolysis. Thus, an electrolyzed water having a stable effective chlorine concentration can be efficiently produced. Furthermore, an electrolytic stock solution 12 having a stable concentration can be supplied to the electrolysis unit 5, which suppresses the degradation of the electrolysis ability of the electrolysis electrode pair 1 in the electrolysis unit 5. Furthermore, in the electrolytic stock solution 12, the concentration of the alkali metal chloride and the concentration of the substance that makes an aqueous solution acidic can be set so that the pH of the electrolyzed water generated is more than 6.5 and less than 8.0.

For example, when the electrolytic stock solution 12 contains sodium chloride and hydrogen chloride (hydrochloric acid), the total concentration of sodium chloride and hydrochloric acid is preferably about 1% or more and about 23% or less because the electrolyzed water generating efficiency decreases if the concentration of sodium chloride is excessively low and a salt easily precipitates if the concentration of sodium chloride is excessively high.

As a result of experiments, it has been found that the ratio of hydrochloric acid/sodium chloride is preferably about 1/20 or more and about ½ or less to control the pH in a desired neutral region and to control the concentration.

In the case where an electrolyzed water containing a high-concentration hypochlorous acid is generated in the electrolysis unit 5 and diluted, the concentration of the hypochlorous acid in the electrolyzed water generated in the electrolysis unit 5 is preferably as high as possible to increase the dilution factor, which decreases the amount of a stock solution. The concentration of the stock solution needs to be increased as the concentration of the hypochlorous acid in the electrolysis unit 5 increases. Otherwise, the generation efficiency decreases. However, if the concentration of the stock solution is excessively high, a salt precipitates and a hydrochloric acid component volatilizes, and thus the concentration easily changes. In an actual operation, an effort of managing the stock solution may be required or an apparatus may be broken.

In an actual operation, therefore, the concentration of the alkali metal chloride is preferably about 5% or more and about 15% or less, and the concentration of the hydrogen chloride is preferably about 0.25% or more and 5% or less.

Herein, when it is expected that the electrolyzed water is not frequently generated and the stock solution is not replenished or exchanged for a long time, the concentrations are preferably decreased overall. The concentration of the alkali metal chloride may be about 0.5% or more and 10% or less, and the concentration of the hydrogen chloride may be about 0.25% or more and 1.0% or less. The specific concentration is determined depending on the situation. For example, if the concentration of the electrolyzed water required is low, the stock solution preferably has a relatively low concentration because the concentration of the stock solution is stabilized for a long time. If the concentration of the electrolyzed water required is high, the stock solution preferably has a relatively high concentration in view of the tradeoff between electrolysis efficiency and stock solution consumption rate.

As a result of thorough studies, it has been found that, for example, when the concentration of the alkali metal chloride is set to about 10% to 20% and the concentration of the hydrogen chloride is set to about 1% to 5%, a high-concentration electrolyzed water in a neutral pH region (pH 6.5 to 8.0, preferably pH 7.0 to 7.5) is generated with a small consumption of stock solution. Typically, the concentration of the alkali metal chloride may be set to about 20% to 15% and the concentration of the hydrogen chloride may be set to about 1.5%. To achieve higher safety, the concentration of the hydrogen chloride may be set to 1% or less. When the concentration of the hydrogen chloride is 1%, the chloride concentration can be set to about 16%. For example, this stock solution was fed to the electrolysis unit 5 described later at a rate of 5 ml/min, electrolyzed at a current of 5 A, and then diluted with tap water at a rate of about 5 L/min. Consequently, an electrolyzed water having a pH of about 7 was obtained. The effective chlorine concentration was about 15 ppm. Specifically, the electrolysis unit 5 had an electrode area of about 20 cm² and an interelectrode distance of about 3 mm, and the electrolysis was performed at a high current density.

Under these conditions, the stock solution can be diluted with a very high dilution factor, and the consumption of stock solution can be decreased. However, it may take a relatively long time (e.g., several minutes) from the start-up to the stabilization of the concentration of the electrolyzed water because the feeding rate is low. When a large amount of electrolyzed water is required, the operation time is several minutes or longer, which poses no particular problems. However, if the operation is intermittently performed within a very short time, the concentration may vary. In this case, preferably, the chloride concentration of the stock solution is decreased and the feeding rate is increased. For example, the concentration of the hydrogen chloride is set to about 0.3%, the concentration of the alkali metal chloride is set to about 6%, and the feeding rate is increased to about 15 ml/min. It is effective for quick start-up that the volume of an electrolytic cell in the electrolysis unit 5 and the volume of a pipe from the outlet of the electrolytic cell to a diluting unit 20 are decreased as much as possible.

Thus, an electrolyzed water suitable for washing can be efficiently generated. When a concentrated electrolytic solution is used as the electrolyte 13 for generating electrolyzed water, it is sufficient that a diluted electrolytic solution supplied to the electrolysis unit 5 has the above concentration.

When the pH is in a neutral region as in the present invention, the pH of the diluted electrolyzed water tends to be dependent on the original pH of dilution water. The dilution water may be pure water, but is normally tap water in terms of cost effectiveness and convenience. Therefore, the pH of an electrolyzed water diluted with tap water is in the range of about pH 5.8 or more and pH 8.6 or less, which is a guideline value of tap water. In reality, the pH is often in the range of about 7.0 to 7.5. A dilution water in which carbon dioxide is dissolved as a result of contact with air for some time and groundwater may have a pH of 7 or less. When the pH of the dilution water is extremely outside the neutral region, the pH of a high-concentration electrolyzed water before dilution is adjusted so that the pH of the diluted electrolyzed water is in the neutral region. Specifically, if the dilution water has an excessively low pH, the amount of the stock solution electrolyzed (effective electrolysis time (inversely proportional to the feeding rate of the stock solution) or current) is increased, the amount of an acid contained in the stock solution is decreased, or both of them are performed to increase the pH of a high-concentration electrolyzed water generated in the electrolysis unit. If the dilution water has an excessively high pH, the amount of the stock solution electrolyzed (effective electrolysis time or current) is decreased, the amount of an acid contained in the stock solution is increased, or both of them are performed to decrease the pH of a high-concentration electrolyzed water generated in the electrolysis unit.

Also in the case where potassium chloride is used instead of sodium chloride, a desired electrolyzed water can be generated in substantially the same concentration range. To be precise, the same weight percentage does not correspond to the same number of moles because of the difference in atomic weight between sodium and potassium, and thus the weight percentage may be converted to molarity. However, for example, the difference in electric conductivity leads to a difference in electrolysis efficiency, and therefore both of the electrolyzed waters are not exactly the same. Nevertheless, even if the concentrations of the boundary conditions differ by about 10% to 20%, the ideal value for the stock solution can be determined by suitably adjusting the concentration within substantially the same concentration range. Furthermore, the difference in pH of a high-concentration electrolyzed water in the electrolysis unit due to the above difference is decreased after dilution with dilution water. In reality, therefore, the difference is negligible, is small compared with the variation in pH of dilution water such as tap water, or can be eliminated by controlling the electrolysis conditions and/or the feeding rate of the stock solution.

When the concentration of the alkali metal chloride in the electrolytic solution supplied to the electrolysis unit 5 is increased, the current density between the electrolysis electrode pair 1 can be increased, which improves the electrolysis efficiency of the electrolysis unit 5. Furthermore, the life characteristics of the electrolysis electrode pair 1 can be improved. Since the electrolysis can be performed at a high current density, the size of the electrolysis electrode pair 1 can be decreased. If the concentration of the alkali metal chloride in the electrolytic solution or the electrolytic stock solution 12 supplied to the electrolysis unit 5 exceeds 20%, the alkali metal chloride tends to precipitate, for example. Therefore, the concentration of the alkali metal chloride is preferably 20% or less.

Although the electrolytic stock solution 12 is supplied to the electrolysis unit 5 with the pump 8 in the electrolyzed water generating device 30 illustrated in FIG. 1, the electrolytic solution tank 7 may be disposed at a position higher than that of the electrolysis unit 5 to supply the electrolytic stock solution 12 to the electrolysis unit 5 by gravity. Alternatively, the electrolytic stock solution 12 may be supplied to the electrolysis unit 5 using a Venturi effect produced by flow of dilution water flowing through an electrolyzed water diluting unit 20.

To facilitate the dissolution of chlorine gas generated, the pressure in the electrolysis unit 5 is preferably increased, but the increase in the pressure may cause liquid leakage. As long as chlorine can be converted into hypochlorous acid before reaching a flow-out port 15, a negative pressure is preferably applied to suppress the leakage of a high-concentration electrolyzed water and gases from the electrolysis unit 5. For example, when a suctioning effect such as a Venturi effect is used, a negative pressure can be applied to the electrolysis unit 5. However, an excessively high negative pressure may inhibit the conversion of chlorine into hypochlorous acid or may generate a large amount of air bubbles. In an extreme case, the boiling point of the aqueous solution decreases, which causes boiling or the like. Therefore, when a negative pressure is applied, the gage pressure is preferably in the range of −0.03 MPa or more and 0.00 MPa or less.

### 3. Electrolysis Unit

The electrolysis unit 5 includes an electrolysis electrode pair 1 including an anode 3 and a cathode 4. The electrolysis electrode pair 1 is provided so that an aqueous solution of the electrolyte 13 for generating electrolyzed water supplied from the electrolytic solution supplying unit 10 flows between the anode 3 and the cathode 4. The electrolysis electrode pair 1 is also provided so that a voltage can be applied between the anode 3 and the cathode 4. Thus, the aqueous solution of the electrolyte 13 for generating electrolyzed water can be electrolyzed, and an electrolyzed water containing hypochlorous acid, a hypochlorite, and an alkali metal chloride can be generated.

For example, it is believed that anode reactions represented by reaction formulae (1) to (3) and a cathode reaction represented by reaction formula (4) proceed in the electrolysis performed in the electrolysis unit 5.

2Cl⁻→Cl₂+2e⁻ (1)

Cl₂+H₂O→HCl+HClO (2)

H₂O→½O₂+2H⁺+2e⁻ (3)

2H₂O+2e⁻→H₂+2OH⁻ (4)

Herein, when an aqueous solution containing an alkali metal chloride is electrolyzed, a hypochlorite such as sodium hypochlorite or potassium hypochlorite is produced, which may impart alkalinity to the electrolyzed water 18. In this embodiment, however, the electrolyte 13 for generating electrolyzed water contains a “substance that makes an aqueous solution acidic” and thus the electrolyzed water 18 is substantially neutral.

The electrolysis unit 5 may include a flow inlet through which an aqueous solution supplied from the electrolytic solution supplying unit 10 flows in and a flow outlet through which an electrolyzed water 18 generated through electrolysis using the electrolysis electrode pair 1 flows out. Thus, the electrolyzed water can be continuously produced by the electrolysis unit 5. The electrolyzed water 18 that has flowed out through the flow outlet may directly flow out through a flow-out port 15 or may flow into the electrolyzed water diluting unit 20. When the electrolyzed water 18 is caused to directly flow out through the flow-out port 15, the electrolysis unit 5 generates an electrolyzed water 18 having a pH of more than 6.5 and less than 8.0. The pH of the electrolyzed water can be adjusted by controlling, for example, the ratio and concentrations of the alkali metal chloride and the substance that makes an aqueous solution acidic in the electrolyte 13 for generating electrolyzed water, the amount of the aqueous solution supplied to the electrolysis unit 5, and the power consumption of the electrolysis electrode pair 1.

When the electrolyzed water 18 is diluted with water by the electrolyzed water diluting unit 20, the electrolyzed water 18 generated by the electrolysis unit 5 may have a pH of 6.5 or less or 8 or more. However, before the electrolyzed water 18 diluted with water by the electrolyzed water diluting unit 20 flows out through the flow-out port 15, the pH of the electrolyzed water 18 is adjusted to more than 6.5 and less than 8.0.

The anode 3 and the cathode 4 may each have a plate shape. The anode 3 and the cathode 4 may be provided so as to face each other without a diaphragm. This decreases the interelectrode distance and improves the electrolysis efficiency. The anode 3 and the cathode 4 may be disposed in substantially parallel so that the interelectrode distance is 1 mm to 5 mm.

The electrolysis electrode pair 1 may be provided so that a single anode 3 and a single cathode 4 face each other, so that anodes 3 and cathodes 4 are alternately stacked on top of each other with a spacing, or so that a plurality of electrodes are stacked and an intermediate electrode has one surface serving as an anode 3 and the other surface serving as a cathode 4.

Alternatively, the electrolysis electrode pair 1 may be disposed so as to incline with respect to the vertical direction so that the anode 3 is located on the upper side and so that an aqueous solution supplied from the electrolytic solution supplying unit 10 flows between the anode 3 and the cathode 4 from the lower side toward the upper side. As a result of a flow of a fluid caused by the floating of air bubbles generated at the cathode 4, a fluid around the cathode 4 and a fluid around the anode 3 can be stirred and mixed, which facilitates the electrode reaction at the anode 3. Thus, an electrolyzed water having a high effective chlorine concentration can be generated.

Except for the case where the feeding rate is excessively low (specifically, a low flow velocity at which it takes about 20 minutes to allow a fluid to pass through a portion between electrodes), when the electrolysis electrode pair 1 is inclined so that the cathode is located on the upper side, the effective chlorine concentration tends to decrease as the inclination angle (an inclination angle with respect to the vertical direction, the same applies hereafter) increases. When the electrolysis electrode pair 1 is inclined so that the anode is located on the upper side, the effective chlorine concentration is equal to that in the case of the vertical direction or is improved by about 10% to 20% at maximum. At an inclination angle of up to about 50°, the generation ability is equal to that in the case of the vertical direction.

When the flow outlet has a bent structure as illustrated in FIG. 1, at an inclination angle of up to about 80°, the generation ability is equal to that in the case of the vertical direction. That is, the flow outlet does not extend in a direction parallel to a flow between the electrodes of the electrolysis unit 5, unlike in the related art. The flow outlet is preferably provided so that the direction of the flow is changed to an upward direction when the electrode pair is inclined. Since the electrode pair is inclined so that the anode is located on the upper side in FIG. 1, the flow outlet is preferably provided in a portion bent toward the anode (in a portion bent at an angle of 90° in FIG. 1). In particular, when the electrode pair is inclined at an angle of 45° or more, the flow outlet preferably has such a structure. At an inclination angle of 50° or more and 80° or less, the effective chlorine concentration is improved in this structure compared with the case where a casing having a known flow inlet/outlet is inclined. When the feeding rate is high, the advantage is reduced, but placing the anode on the upper side is still better than placing the cathode on the upper side.

This allows a decrease in the height of the entire generating device. In a known electrolyzed water generating device, the electrode pair of the electrolysis unit is disposed in a substantially vertical direction. Therefore, the minimum size of the generating device is dependent on the height of the electrolysis unit, which is a design constraint. Typically, in the case where an electrolysis unit having a longitudinal, substantially box shape or cylindrical shape (including a cylindroid) is designed so as to have a minimum volume, “area of bottom surface maximum area of projected side surface” needs to be satisfied.

For example, when the electrode pair is simply inclined at an angle of 60°, the height can be decreased to about a half. Furthermore, since the electrode pair can be inclined at an angle of 45° or more, the generating device can be simply designed to have a structure in which a known generating device is put into a sideways position as long as no influence is exerted on other constituent components. That is, the generating device can be provided so that the electrode pair of the electrolysis unit satisfies “area of bottom surface maximum area of projected side surface”. The generating device that satisfies the above requirements is less likely to topple and is safe. From another view point, the generating device shows its ability even when inclined at a large angle of 0 to 80°. Therefore, the generating device can also be used in an oblique manner at a place where a level surface is not easily provided. The generating device is excellent in terms of convenience.

For example, the electrolysis electrode pair 1 may include an electrode (referred to as a Ti electrode) formed of a titanium plate and an electrode (referred to as an Ir-coated Ti electrode) obtained by coating a titanium plate with iridium oxide by a sintering method. A power supply circuit and the electrolysis electrode pair 1 can be connected to each other so that the Ti electrode serves as a cathode 4 and the Ir-coated Ti electrode serves as an anode 3.

### 4. Electrolyzed Water Diluting Unit and Flow-Out Port

The electrolyzed water diluting unit 20 is provided so as to dilute the electrolyzed water 18 generated by the electrolysis unit 5 with water and supply the diluted electrolyzed water to the flow-out port 15. Thus, an electrolyzed water 18 having an effective chlorine concentration of 10 ppm or more and 100 ppm or less can be generated and can be caused to flow out through the flow-out port 15. Furthermore, the pH of the electrolyzed water 18 that flows out through the flow-out port 15 can be adjusted to more than 6.5 and less than 8.0.

When the electrolyzed water 18 generated by the electrolysis unit 5 is diluted with water by the electrolyzed water diluting unit 20, the amount of the electrolyzed water 18 produced can be increased. The dilution water may be, for example, tap water. When the electrolyzed water diluting unit 20 is provided, the amount of dilution water can be changed, and thus the effective chlorine concentration of the electrolyzed water 18 can be easily changed.

The flow-out port 15 is a portion through which the electrolyzed water 18 generated by the electrolyzed water generating device 30 is caused to flow out. The flow-out port 15 may be a portion in which the electrolyzed water generating device 30 and a water pipe are connected to each other or a portion through which the generated electrolyzed water 18 is discharged to the outside.

The electrolyzed water diluting unit 20 may be provided so that the flow of the electrolyzed water 18 generated by the electrolysis unit 5 joins the flow of dilution water. In this case, the electrolyzed water diluting unit 20 can be provided so that the flow of the electrolyzed water 18 generated by the electrolysis unit 5 joins the flow of water flowing in a substantially horizontal direction. This increases the effective chlorine concentration of the electrolyzed water 18 that flows out through the flow-out port 15. The electrolyzed water diluting unit 20 may also be provided so that the electrolyzed water 18 generated by the electrolysis unit 5 is sucked using a Venturi effect produced by flow of dilution water.

The electrolyzed water diluting unit 20 may also be provided so that dilution is performed in a dilution tank into which the electrolyzed water 18 generated by the electrolysis unit 5 and the dilution water flow.

For example, in the electrolyzed water generating device 30 illustrated in FIG. 1, tap water supplied from a faucet flows in through a solenoid-controlled valve 22. The flow of the electrolyzed water 18 generated by the electrolysis unit 5 joins the flow of the tap water in the electrolyzed water diluting unit 20.

### 5. Stirring Unit

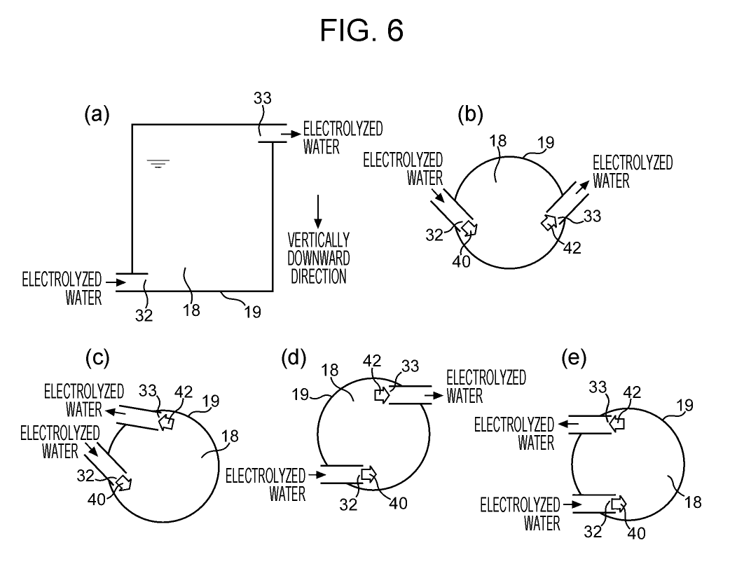

The electrolyzed water generating device 30 may include a stirring unit 19. FIGS. 5(a) to 5(c) are schematic sectional views of stirring units 19 included in the electrolyzed water generating device 30 according to this embodiment. FIG. 5(d) is a schematic sectional view of an air bubble dividing unit 35 included in the stirring unit 19. FIG. 6(a) is a schematic vertical sectional view of the stirring unit 19. FIGS. 6(b) to 6(e) are schematic views obtained by projecting the stirring units 19 in the vertical direction and illustrate the horizontal positional relationship between a flow inlet 32 and a flow outlet 33 included in the stirring unit 19 illustrated in FIG. 6(a).

The stirring unit 19 is provided so that the electrolyzed water 18 diluted by the electrolyzed water diluting unit 20 flows into the stirring unit 19, and the electrolyzed water 18 that has flowed out from the stirring unit 19 is supplied to the flow-out port 15. When such a stirring unit 19 is provided, the pH and effective chlorine concentration of the electrolyzed water that flows out through the flow-out port 15 can be stabilized. Consequently, an electrolyzed water 18 having stable quality can be generated. The stirring unit 19 may be a water tank that generates a turbulent flow or a stirring tank equipped with a stirring bar.

The stirring unit 19 may be provided so that the electrolyzed water 18 containing a chlorine gas that has not been completely converted into a hypochlorite in the electrolysis unit 5 and the diluting unit 20 flows into the stirring unit 19. By stirring the electrolyzed water 18, the chlorine gas is dissolved in the electrolyzed water and converted into hypochlorous acid.

In particular, in the case where chlorine gas is possibly released to a space without being dissolved and converted, the stirring unit 19 of the present invention is preferably installed. Examples of the case include a case where the stock solution has a relatively low pH, a case where the concentration of hypochlorous acid produced in the electrolysis unit 5 is high, a case where the electrolyzed water generated has a relatively low pH, a case where the electrolyzed water generated has a high concentration, a case where a pipe that connects the electrolysis unit 5 and the diluting unit 20 is relatively short, and a case where the distance from the diluting unit 20 to a release point for the space (flow-out port 15 or the other open end of a series of pipes such as hoses connected to the flow-out port 15) is relatively short.

The stirring unit 19 may include a flow inlet 32 into which the electrolyzed water 18 generated by the electrolysis unit 5 flows and a flow outlet 33 through which the electrolyzed water 18 flows out from the stirring unit 19. The flow outlet 33 may be provided in an upper portion of the stirring unit 19 to prevent gas from being easily accumulated. The flow inlet 32 may be provided below the flow outlet 33.

In the case where an unintended gas is contained that is not desired to be mixed in, dissolved in, or reacted with the electrolyzed water 18 flowing into the stirring unit 19, such an unintended gas is preferably quickly released to the outside of the stirring unit 19. Therefore, the flow outlet 33 is preferably provided in an upper portion of the stirring unit 19 as illustrated in FIGS. 5(a) to 5(c) and FIG. 6(a). This suppresses accumulation of unintended gas in the stirring unit 19, and thus suppresses a decrease in the stirring function of the stirring unit 19.

Examples of the relationship between the flow inlet 32 and the flow outlet 33 include (1) a relationship in which a flux direction 40 of the electrolyzed water 18 that flows in through the flow inlet 32 is not parallel to a flux direction 42 of the electrolyzed water 18 that flows toward the flow outlet 33, (2) a relationship in which, when projected in a vertical direction, a flux direction 40 does not overlap a flux direction 42, and (3) a relationship in which an obstacle (barrier 37) is present on a line segment that connects the flow inlet 32 and the flow outlet 33.

In such a configuration, a complicated turbulent flow is formed in the stirring unit 19, and the electrolyzed water and the chlorine gas can be mixed with each other even when the stirring unit 19 is small. Consequently, the dissolution and reaction can be facilitated. The relationship (1) is, for example, a relationship between the flow inlet 32 and the flow outlet 33 of the stirring units 19 illustrated in FIGS. 5(a) and 5(c) and FIGS. 6(b) and 6(c). The relationship (2) is, for example, a relationship between the flow inlet 32 and the flow outlet 33 of the stirring units 19 illustrated in FIGS. 6(b) to 6(e). The relationship (3) is, for example, a relationship between the flow inlet 32 and the flow outlet 33 of the stirring unit 19 illustrated in FIG. 5(b).

The presence of the stirring unit 19 provides a very simple structure without a gas storage unit and a circulation path. Consequently, the gas-liquid contact area and contact time can be increased, the local pressure of a gas-liquid interface can be increased because of a large change in momentum, and reaggregated large air bubbles can be quickly divided into small air bubbles. Thus, the mixing, dissolution, reaction, and the like between gas and liquid can be efficiently caused.

Furthermore, the gas can be prevented from being stored or accumulated in the stirring unit 19 as much as possible. This suppresses a change in the constituent concentration in the water, that is, a variation in the concentration caused by a change in the amount of gases stored or accumulated and a change in the constituent concentration over time. Since the stirring unit 19 is small and has a small storage portion, the time constant of a state in the stirring unit 19 is small and the rise/fall time is short. Therefore, a high effect is produced when the stirring unit 19 is employed in a device that continuously generates a certain fluid such as a device that generates hypochlorous acid water through electrolysis, in particular, a device frequently operated on an intermittent basis or a device in which each operation time is short. Thus, a device having a small variation in concentration can be provided.

For example, when an aqueous solution containing a chloride is electrolyzed in the electrolysis unit 5 to produce hypochlorous acid, hydrogen is generated in addition to chlorine that needs to be subjected to mixing, dissolution, and reaction to produce hypochlorous acid. In this case, hydrogen molecules, which are relatively not easily dissolved in water, immediately gasify, and the ratio of hydrogen gas sometimes increases in the stirring unit 19 when the stirring unit 19 has a storage portion. If hydrogen gas is accumulated in the stirring unit 19, a function of dissolving chlorine gas in the electrolyzed water 18 in the stirring unit 19 is degraded. Furthermore, hydrogen gas is a combustible gas. In the case where hydrogen gas is accidentally released to an ignition source in a stroke because of some trouble, ignition and, in the worst case, explosion are likely to occur. Therefore, the flow outlet 33 is provided in an upper portion of the stirring unit 19 to prevent gas from being easily accumulated, and the gas in the stirring unit 19 is discharged to an open space at all times. Consequently, hydrogen gas, which is much lighter than air, is immediately diffused and diluted by air. The hydrogen gas concentration falls below the explosion limit, and ignition is less likely to occur. Moreover, the gas is released constantly and thus water and a trace amount of hydrogen gas are intermittently released. Therefore, even if an ignition source is present at a position very close to a release outlet and a trace amount of hydrogen gas burns, water arrives instantly and thus the combustion completes in a short time. There are substantially no risks of fire or explosion.

The flow inlet 32 is preferably provided in at least a bottom half of the stirring unit 19 so that the electrolyzed water 18 flows in downward. Thus, the electrolyzed water 18 that has flowed into the stirring unit 19 downward turns and moves up in the stirring unit 19, and thus air bubbles can travel a long path from the flow inlet 32 to the flow outlet 33. Since a change in momentum is large, a stirring effect can be increased on both gas and liquid. Thus, the mixing, dissolution, and reaction of chlorine gas in the electrolyzed water 18 can be facilitated.

Herein, the stirring unit 19 and the pipe connected to the stirring unit 19 are distinguished by the flow inlet 32 and the flow outlet 33. On the assumption that a typical pipe has a substantially constant diameter and cross-sectional area, the flow inlet/outlet can be defined as a boundary portion between the pipe and a space having a diameter or cross-sectional area different from the diameter or cross-sectional area of the pipe. Alternatively, the flow inlet/outlet can be defined as a boundary portion in which the average flow velocity of a liquid flowing at a constant flow rate is different from that of a liquid in the pipe. For example, when a pipe having a large internal diameter is intentionally inserted in the middle of the pipe, the connecting portion can be regarded as a flow inlet/outlet and the thick pipe can be regarded as a stirring unit.

The stirring unit 19 preferably includes an air bubble dividing unit 35 at the flow inlet 32. The air bubble dividing unit 35 can be provided, for example, as illustrated in FIGS. 5(a), 5(c), and 5(d). The air bubble dividing unit 35 has a mesh-like shape or a shape similar to the mesh-like shape. When the air bubble dividing unit 35 is provided at the flow inlet 32, air bubbles 45 that flow in through the flow inlet 32 together with the electrolyzed water 18 can be minutely divided. Therefore, the total surface area of the gas-liquid interface, that is, the contact area can be increased, which facilitates the dissolution and reaction of chlorine gas. In this structure in which the air bubbles 45 are forced out downward, pressure is applied to the air bubbles 45 and the dissolution and reaction of the air bubbles 45 can be facilitated. The air bubble dividing unit 35 may have, for example, a perforated shape such as a mesh-like shape, a shape with many punched holes, or a slit shape, or a lattice shape.

Furthermore, the stirring unit 19 may have a structure in which water is retained when the apparatus is stopped. It is normally common sense that the retention of water is avoided as much as possible in water pipes to prevent the propagation of germs. However, in the case where water is retained, even if a high-concentration hypochlorous acid water left in the electrolysis unit when electrolysis is stopped flows out to the diluting unit 20 for some reason, the high-concentration hypochlorous acid water can be prevented from flowing out to a space through the pipe. Obviously, the stock solution can be supplied and discharged without electrolysis to remove the high-concentration hypochlorous acid from the electrolysis unit 5, but the stock solution is wasted. Therefore, when the apparatus is frequently used, the stock solution preferably converted into hypochlorous acid water efficiently. When the apparatus is stopped for a long time, a high-concentration hypochlorous acid water is preferably prevented from being left in the electrolysis unit 5.

### 6. Control System

The electrolyzed water generating device 30 may have a control system illustrated in FIG. 2. For example, a control/power supply circuit can be connected to a power supply circuit for electrolysis, a voltmeter or an ammeter, a water level sensor for an electrolytic solution tank, a solenoid-controlled valve 22, a flowmeter for electrolyzed water that flows out through the flow-out port 15, and a control/display unit. Thus, a user of the electrolyzed water generating device 30 can control the electrolyzed water generating device 30 and check the state of the electrolyzed water generating device 30 using the control/display unit.

A safety device performs automatic stop and error indication using the above-described instruments and sensors. In this embodiment, error indication is given when the electrolysis unit is abnormal (specifically, detection of voltage in a constant-current drive or detection of current in a constant-voltage drive), the dilution water is abnormal (specifically, detection of the amount of water or possibly detection of water pressure when the outlet is an open end), and the stock solution runs out (specifically, detection of water level or weight in the tank), and automatic stop is performed.

### Second Embodiment

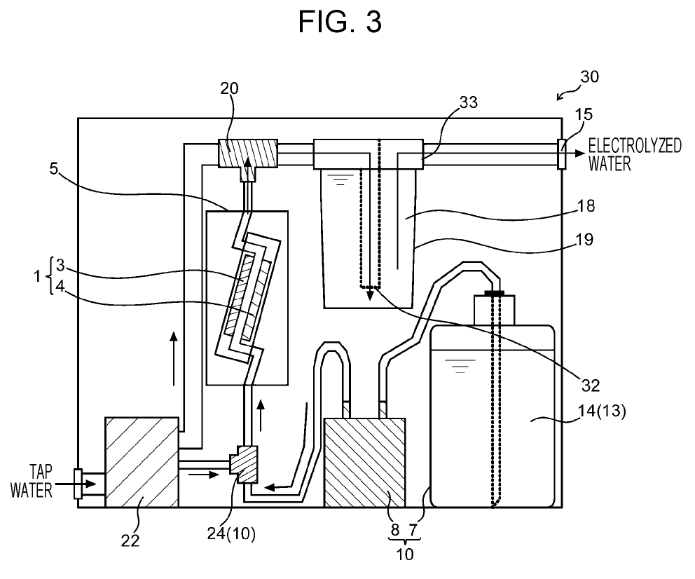

FIG. 3 is a schematic sectional view of an electrolyzed water generating device 30 according to a second embodiment. In the second embodiment, the electrolytic solution supplying unit 10 includes an electrolytic solution diluting unit 24. A concentrated electrolytic solution 14 serving as an electrolyte 13 for generating electrolyzed water is stored in the electrolytic solution tank 7. The electrolytic solution supplying unit 10 dilutes the concentrated electrolytic solution 14 with tap water in the electrolytic solution diluting unit 24 and supplies an electrolytic solution having an appropriate concentration to an electrolysis unit 5.

By employing such a configuration, the volume of the electrolytic solution tank 7 can be decreased, which reduces the size of the electrolyzed water generating device 30. The electrolyte for generating electrolyzed water is also easily replenished to the electrolyzed water generating device 30.

The description in the first embodiment applies to the second embodiment unless a contradiction arises.

### Third Embodiment

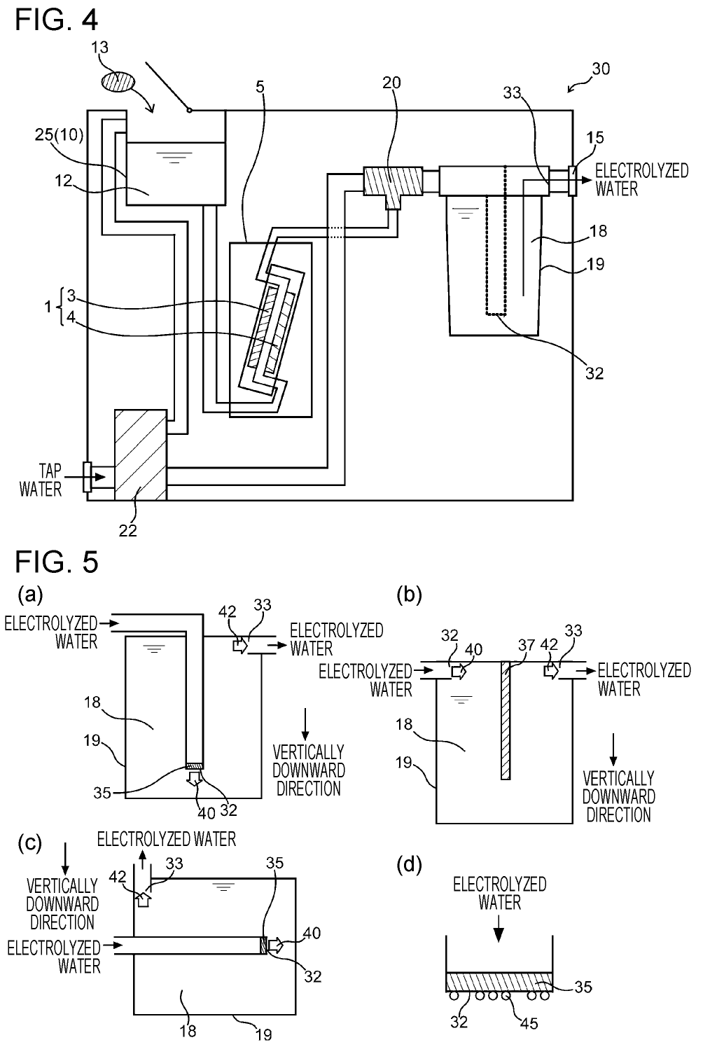

FIG. 4 is a schematic sectional view of an electrolyzed water generating device 30 according to a third embodiment. In the third embodiment, the electrolytic solution supplying unit 10 includes an electrolytic solution preparing unit 25. The electrolytic solution preparing unit 25 is provided so that an electrolyte 13 for generating electrolyzed water can be put into the electrolytic solution preparing unit 25. The electrolyte 13 for generating electrolyzed water to be put into the electrolytic solution preparing unit 25 may be a concentrated solution or a powder. An example of a powdery electrolyte 13 for generating electrolyzed water is a mixed powder of sodium chloride or potassium chloride and citric acid.

The electrolytic solution preparing unit 25 is connected to a solenoid-controlled valve 22 so that water can be supplied to the electrolytic solution preparing unit 25. The electrolytic solution supplying unit 10 is provided so that an electrolytic solution is prepared by diluting the electrolyte 13 for generating electrolyzed water with water or dissolving the electrolyte 13 in water in the electrolytic solution preparing unit 25 and the prepared electrolytic solution is supplied to the electrolysis unit 5. The electrolytic solution preparing unit 25 may be provided so that a uniform electrolytic solution can be prepared using a stirring bar or so that a uniform electrolytic solution can be prepared using a flow of water that flows into the electrolytic solution preparing unit 25.

In this configuration, the electrolyzed water generating device 30 does not necessarily include an electrolytic solution tank 7 and thus the size of the electrolyzed water generating device 30 can be reduced. The electrolyzed water generating device 30 can be incorporated into a washing machine or the like. The electrolyte 13 for generating electrolyzed water is also easily supplied to the electrolyzed water generating device 30.

The description of the first embodiment applies to the third embodiment unless a contradiction arises.

### Electrolyzed Water Generation Experiment 1

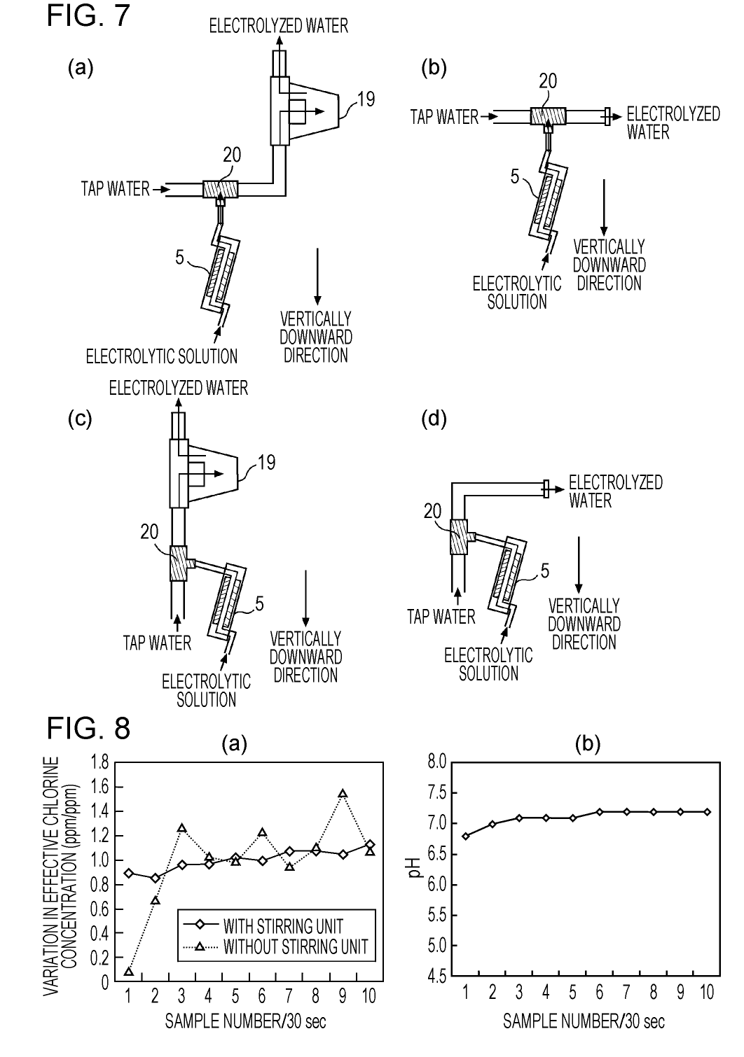

Electrolyzed water generating devices illustrated in FIGS. 7(a) to 7(d) were produced, and an experiment of generating an electrolyzed water was performed. The electrolyzed water generating devices (a) to (d) were different from each other in terms of the direction in which tap water flowed through the electrolyzed water diluting unit 20 and the presence or absence of the stirring unit 19. The electrolysis electrode pair 1 included in the electrolysis unit 5 was a titanium-ruthenium electrode pair. The electrolytic solution supplied to the electrolysis unit 5 was a mixed aqueous solution of NaCl+HCl, and the amount of the electrolytic solution supplied to the electrolysis unit 5 was 5 ml/min. A current of 6.2 A was applied to the electrolysis electrode pair 1. The amount of tap water flowing through the electrolyzed water diluting unit 20 was about 5 L/min. The stirring unit 19 was a part of a strainer that satisfies the following requirements. In the stirring unit 19, the outlet was provided in an upper portion of the stirring unit 19 to prevent gas from being easily accumulated. The inlet was located at the same level as that of the outlet or below the outlet. The relationship between the inlet and the outlet was a relationship in which a flux direction at the inlet is not parallel to a flux direction at the outlet, a relationship in which, when projected in a vertical direction, flux directions do not overlap each other, or a relationship in which an obstacle is present on a line segment that connects the inlet and the outlet.

In the stirring unit 19 used in this experiment, an electrolyzed water diluted with tap water flows in from a middle portion of the stirring unit 19 in a substantially horizontal flux direction. The outlet is located in an upper portion of the stirring unit 19. The flux direction at the outlet has a substantially upward flux component. That is, the flux direction at the inlet and the flux direction at the outlet are not parallel to each other.

Under these conditions, an electrolyzed water was generated. An electrolyzed water generated ten minutes after the start of electrolysis was sampled, and the effective chlorine concentration was measured. The flow rate of the generated electrolyzed water was also measured.

Table 1 shows the measurement results of the electrolyzed water generation experiment 1. It was found that the effective chlorine concentration of the electrolyzed water generated in the generating devices (a) and (c) including the stirring unit 19 was higher than the effective chlorine concentration of the electrolyzed water generated in the generating devices (b) and (d). This may be because a reaction in which chlorine gas is converted into hypochlorous acid was caused to proceed by providing the stirring unit 19. It was also found that the effective chlorine concentration of the electrolyzed water generated in the generating device in which tap water was caused to flow through the diluting unit 20 in a horizontal direction was higher than that of the electrolyzed water generated in the generating device in which tap water was caused to flow in a vertically upward direction. Although the reason for this is unclear, this may be because air bubbles have a property of moving in a vertically upward direction in liquid and thus impart a resistance to the flow of tap water in a horizontal direction rather than in a vertical direction. Therefore, pressure is easily applied to air bubbles and the stream is easily disturbed, and thus unconverted chlorine gas is easily converted into hypochlorous acid.

The flow of tap water may be from the upper side to the lower side. In this case, however, if the stream of tap water is weak, air bubbles possibly flow backward. Consequently, for example, air bubbles are accumulated, which may increase variations in the flow rate and concentration of tap water.

Therefore, dilution water that has flowed through the diluting unit 20 desirably flows in a horizontal direction until unconverted chlorine gas is converted into hypochlorous acid. The dilution water preferably flows in a horizontal direction at a position as closely as possible to the diluting unit 20 because the conversion quickly occurs. Therefore, most preferably, tap water flows in a horizontal direction at the diluting unit 20.

The experiment was also performed using a titanium-iridium electrode pair for confirmation. The same tendency was observed.

TABLE 1

| | | | Effective | | |
|---|---|---|---|---|---|
| | | | chlorine | | Measured flow |
| | Direction of | | concentration | | rate of |
| | tap water in | Stirring | of electrolyzed | | electrolyzed |
| | diluting unit | unit | water | | water (L/min) |
| Generating | Horizontal | Presence | 13 | ppm | 4.80 |
| device (a) | direction | | | | |
| Generating | Horizontal | Absence | 8.7 | ppm | 4.87 |
| device (b) | direction | | | | |
| Generating | Vertically | Presence | 10.5 | ppm | 4.69 |
| device (c) | upward | | | | |
| | direction | | | | |
| Generating | Vertically | Absence | 6.7 | ppm | 4.84 |
| device (d) | upward | | | | |
| | direction | | | | |

### Electrolyzed Water Generation Experiment 2

An electrolyzed water generating device 30 illustrated in FIG. 1 was produced and an experiment of generating an electrolyzed water was performed.

In the electrolysis electrode pair 1 included in the electrolysis unit 5, the anode was a Ti plate including an iridium oxide film and the cathode was a Ti plate. The electrolytic solution supplied to the electrolysis unit 5 was a mixed aqueous solution of NaCl+HCl, and the amount of the electrolytic solution supplied to the electrolysis unit 5 was 20 ml/min. A constant voltage of 5 V with an upper limit current of 6.2 A was applied to the electrolysis electrode pair 1. The amount of tap water flowing through the electrolyzed water diluting unit 20 was about 5 L/min. The stirring unit 19 was a part of a strainer. In the stirring unit 19 used in this experiment, the electrolyzed water diluted with tap water flows in from a flow inlet 32 provided in a middle portion of the stirring unit 19 in a substantially downward flux direction. A flow outlet 33 is provided in an upper portion of the stirring unit 19, and the flux direction toward the flow outlet 33 has a substantially upward or horizontal flux component. That is, the flux direction at the flow inlet 32 and the flux direction at the flow outlet 33 are not parallel to each other.

In this experiment, since the flow rate of tap water is high and thus the flow velocity is high, the accumulation of air bubbles substantially does not occur on route. If the flow velocity of tap water is low and air bubbles may be accumulated, the flux direction at a pipe just before the dilution water may be a horizontal direction, an upward direction, or a direction between the horizontal direction and the upward direction. The inlet for the stirring unit may be provided in the same direction as the flux direction, and the outlet may be provided above the inlet and so that the flux direction at the inlet does not match the flux direction at the outlet. Alternatively, an obstacle may be disposed between the inlet and the outlet. For example, the stirring unit used in the electrolyzed water generation experiment 1 satisfies the structural requirements.

An electrolyzed water was generated under these conditions. The electrolyzed water was sampled every 30 seconds and the effective chlorine concentration and pH were measured.

FIGS. 8(a) and 8(b) illustrate the measurement results of the electrolyzed water generation experiment 2. FIG. 8(a) also illustrates the measurement result obtained when an electrolyzed water was generated using an electrolyzed water generating device not equipped with the stirring unit 19. The effective chlorine concentration in FIG. 8(a) is determined by dividing the measured effective chlorine concentrations by the average of the effective chlorine concentrations.

As illustrated in FIG. 8(a), it was found that the variation in the effective chlorine concentration of the generated electrolyzed water could be suppressed and the effective chlorine concentration could be stabilized by providing the stirring unit 19. Furthermore, the effective chlorine concentration could be quickly increased. As illustrated in FIG. 8(b), it was found that the pH of the generated electrolyzed water was stabilized at about 6.8 to 7.2. In particular, except for the first sampling at the start-up during which an unelectrolyzed stock solution component tends to be contained at the beginning of electrolysis, the pH after the second sampling (after 1 minute) was stabilized at about 7.0 to 7.2 and the pH after the third sampling (after 1.5 minutes) was considerably stabilized at 7.1 to 7.2. Therefore, an electrolyzed water having stable quality could be generated in the produced electrolyzed water generating device.

### Disinfection Experiment

Electrolyzed waters (HCl+NaCl electrolyzed waters (1) to (5)) having an effective chlorine concentration of 20 ppm to 600 ppm were generated using the electrolyzed water generating device 30 produced in the electrolyzed water generation experiment 2. The generation conditions of the electrolyzed waters were the same as those of the electrolyzed water generation experiment 2, except for the amount of tap water flowing through the electrolyzed water diluting unit 20. By changing the amount of tap water flowing through the electrolyzed water diluting unit 20, the effective chlorine concentration of the electrolyzed water was adjusted. Hereafter, the concentration of electrolyzed waters and the concentration of bleaching solutions are each an effective chlorine concentration.

A 100%-cotton cloth with sides of 5 cm was put into 100 ml of the generated electrolyzed water and stirred with a stirrer for 3, 10, or 30 minutes to disinfect the cloth. Subsequently, the disinfected cloth was rinsed with 100 ml of tap water for 1 minute and then rinsed again with another 100 ml of tap water for 1 minute. The second rinse water was sampled and a microorganisms test for general live bacteria was performed. In the microorganisms test, 1 ml of rinse water was added to a standard agar medium and left to stand at room temperature for 3 days to culture the general live bacteria, and the number of bacterial colonies generated was determined. The cloth subjected to the disinfection and rinse was dried and whether fading occurred or not was determined with a reflectometer or the like.

For comparison, the treatment solution used for disinfection was changed to tap water, a commercially available 20 ppm to 1000 ppm bleaching solution, and a 50 ppm to 600 ppm NaCl electrolyzed water, and the same experiment was performed. The commercially available bleaching agent was a household chlorinated bleaching agent. The NaCl electrolyzed water was an electrolyzed water generated as an aqueous NaCl solution not containing an acid from the electrolytic solution supplied to the electrolysis unit 5 using an electrolyzed water generating device 30 produced in the electrolyzed water generation experiment 2.

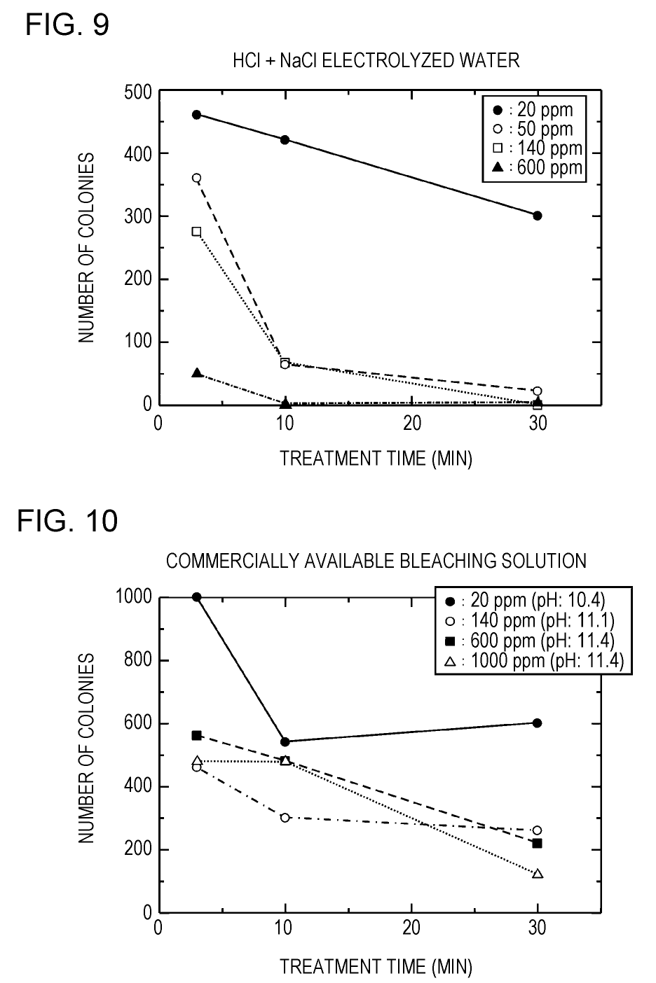

Table 2 and FIGS. 9 to 11 illustrate the results of the disinfection experiment. Table 2 also shows the effective chlorine concentration of the treatment solution used for disinfection. In the experiment in which tap water was used as the treatment solution, the number of bacterial colonies was excessively large and could not be determined (referred to as “many”, the same applies to other tables).

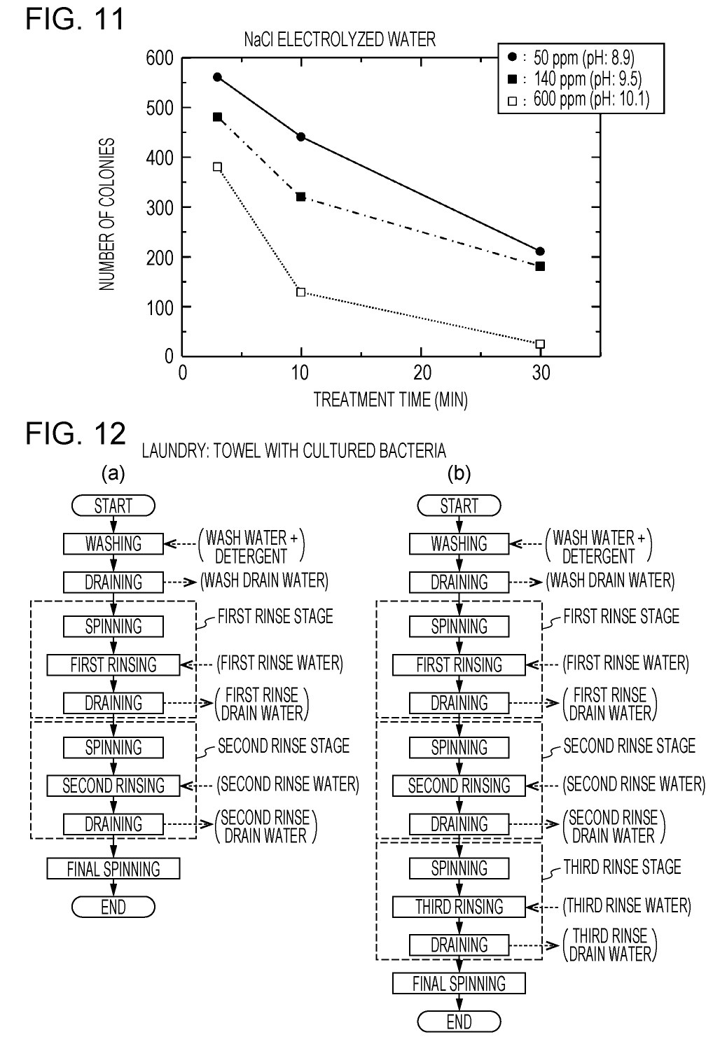

FIG. 9 illustrates the relationship between the disinfection treatment time and the determined number of bacterial colonies in the disinfection experiment using a HCl+NaCl electrolyzed water. FIG. 10 illustrates the relationship between the disinfection treatment time and the determined number of bacterial colonies in the disinfection experiment using a commercially available bleaching solution. FIG. 11 illustrates the relationship between the disinfection treatment time and the determined number of bacterial colonies in the disinfection experiment using a NaCl electrolyzed water.

TABLE 2

| | Effective | | Treatment time: 3 minutes | | Treatment time: 10 minutes | | Treatment time: 30 minutes | |
|---|---|---|---|---|---|---|---|---|
| | chlorine | | | Number of | | Number of | | Number of |
| Treatment solution | concentration | | Fading | colonies | Fading | colonies | Fading | colonies |
| Tap water | 0.3 | ppm | No | many | No | many | No | many |
| HCl + NaCl electrolyzed | 20 | ppm | No | 460 | No | 420 | No | 300 |
| water (1) | | | | | | | | |
| HCl + NaCl electrolyzed | 50 | ppm | No | 360 | No | 64 | No | 22 |
| water (2) | | | | | | | | |
| HCl + NaCl electrolyzed | 140 | ppm | No | 275 | Yes (light) | 67 | Yes (light) | 0 |
| water (3) | | | | | | | | |
| HCl + NaCl electrolyzed | 600 | ppm | Yes (heavy) | 50 | Yes (heavy) | 1 | Yes (heavy) | 4 |
| water (4) | | | | | | | | |
| Commercially available | 20 | ppm | No | 1000 | No | 540 | Yes (light) | 600 |
| bleaching solution (1) | | | | | | | | |
| Commercially available | 140 | ppm | No | 460 | No | 300 | Yes (light) | 260 |
| bleaching solution (2) | | | | | | | | |
| Commercially available | 600 | ppm | No | 560 | Yes (light) | 480 | Yes (light) | 219 |
| bleaching solution (3) | | | | | | | | |
| Commercially available | 1000 | ppm | Yes (light) | 480 | Yes (light) | 480 | Yes (heavy) | 121 |
| bleaching solution (4) | | | | | | | | |
| NaCl electrolyzed water (1) | 50 | ppm | No | 560 | No | 440 | No | 210 |
| NaCl electrolyzed water (2) | 140 | ppm | No | 480 | Yes (light) | 320 | Yes (light) | 180 |
| NaCl electrolyzed water (3) | 600 | ppm | Yes (light) | 380 | Yes (heavy) | 128 | Yes (heavy) | 24 |

The pH of the HCl+NaCl electrolyzed water used for disinfection was about 7.5. The pH of the commercially available bleaching solution was about 10 to 11. The pH of the NaCl electrolyzed water was about 9 to 10. The commercially available bleaching solution was believed to be alkaline because sodium hypochlorite and sodium hydroxide were main solutes. The NaCl electrolyzed water was believed to be alkaline because sodium hypochlorite produced through electrolysis of the aqueous NaCl solution and NaCl were main solutes. The HCl+NaCl electrolyzed water was believed to be neutral because hypochlorous acid, sodium hypochlorite, HCl, and NaCl were main solutes.

As illustrated in FIG. 9, when a 20 ppm HCl+NaCl electrolyzed water was used for disinfection, the number of colonies was 460 in the treatment time of 3 minutes and 300 in the treatment time of 30 minutes.

These numbers of colonies are substantially equal to those obtained in the case where a 140 ppm commercially available bleaching solution in FIG. 10 was used and in the case where a 140 ppm NaCl electrolyzed water in FIG. 11 was used. Thus, the 20 ppm HCl+NaCl electrolyzed water was found to have substantially the same degree of disinfectant properties as the 140 ppm commercially available bleaching solution and the 140 ppm NaCl electrolyzed water. Furthermore, fading was observed on the treated cloth when the cloth was disinfected with a 140 ppm commercially available bleaching solution for 30 minutes and when the cloth was disinfected with a 140 ppm NaCl electrolyzed water for 10 minutes and 30 minutes. In contrast, fading was not observed on the treated cloth when the cloth was disinfected with a 20 ppm HCl+NaCl electrolyzed water.

As illustrated in FIG. 9, when the effective chlorine concentration of the HCl+NaCl electrolyzed water was increased, the determined number of colonies was decreased. In the experiment in which disinfection was performed using a HCl+NaCl electrolyzed water having an effective chlorine concentration of 50 ppm or more for 30 minutes, the determined number of colonies was 50 or less. When disinfection was performed using a HCl+NaCl electrolyzed water having an effective chlorine concentration of 100 ppm or less, fading was not observed on the treated cloth.

Accordingly, it was found that, at an effective chlorine concentration of 100 ppm or less, the HCl+NaCl electrolyzed water had good disinfectant properties and the fading of the treated cloth was suppressed.

### Washing Experiment 1

The electrolyzed water generating device 30 produced in the electrolyzed water generation experiment 2 was connected to a drum washing machine. Half of a towel in which general live bacteria were cultured with cow's milk and 6 kg of clean towels were washed as laundry through the process (double rinsed) illustrated in FIG. 12(a) using a HCl+NaCl electrolyzed water having an effective chlorine concentration of 50 ppm as a rinse water. Furthermore, a first rinse drain water and a second rinse drain water were sampled, and the microorganisms test for general live bacteria was performed to determine the number of colonies. The microorganisms test was performed by the same method as that of the disinfection experiment. The generation conditions of the HC +NaCl electrolyzed water were the same as those of the electrolyzed water generation experiment 2 except for the amount of tap water flowing through the electrolyzed water diluting unit 20, and the effective chlorine concentration of the electrolyzed water was adjusted by changing the amount of tap water flowing through the electrolyzed water diluting unit 20. The wash step was performed with a commercially available laundry detergent using tap water as a wash water as in general washing. The substantial washing time except for the water supply time was changed in accordance with the weight of laundry as in general washing. When 6 kg or more of laundry (towels in this experiment) was put into the machine, the washing time was set to 14 minutes. When the weight of laundry was less than 2 kg as in a washing experiment 2 and the following experiments described below, the washing time was set to 4 minutes.

The same experimental conditions are also employed in the following washing experiments unless a contradiction arises. The electrolyzed waters in the washing experiments are each a HCl+NaCl electrolyzed water.

Table 3 shows the washing conditions of washings 1 to 3 and the determined numbers of colonies.

The numbers of colonies in the first rinse drain water were 0 in both the washings 2 and 3. However, the number of colonies in the second rinse drain water was 16 in the washing 3 whereas the number of colonies was 131 in the washing 2. This showed that, when a large amount of laundry is washed, the rinse time with the electrolyzed water is desirably lengthened.

TABLE 3

| | | First rinse | Second rinse | Specimen 1 | Specimen 2 |
|---|---|---|---|---|---|
| | | water | water | (number of | (number of |
| | Laundry | (rinse time) | (rinse time) | colonies) | colonies) |
| Washing 1 | Cultured towel | Tap water | Tap water | First rinse | Second rinse |
| (Comparative | (half) + | (2 minutes) | (2 minutes) | drain water | drain water |
| Example) | Clean towel (6 kg) | | | (820) | (213) |
| Washing 2 | Cultured towel | 50 ppm | Tap water | First rinse | Second rinse |
| (Example) | (half) + | electrolyzed | (2 minutes) | drain water | drain water |
| | Clean towel (6 kg) | water | | (0) | (131) |
| | | (2 minutes) | | | |
| Washing 3 | Cultured towel | 50 ppm | Tap water | First rinse | Second rinse |
| (Example) | (half) + | electrolyzed | (2 minutes) | drain water | drain water |
| | Clean towel (6 kg) | water | | (0) | (16) |
| | | (10 minutes) | | | |

### Washing Experiment 2

In the washing experiment 2, half of a towel in which general live bacteria were cultured with cow's milk was used as laundry. Washing was performed through the process illustrated in FIG. 12(a) using a 50 ppm electrolyzed water as a first rinse water, and the microorganisms test was performed on the rinse drain water. Furthermore, washing experiments of Comparative Examples were performed in which tap water and a commercially available household chlorinated bleaching solution were used as a rinse water.

Table 4 shows the washing conditions of washings 4 to 8 and the determined numbers of colonies.

In the washings 5 and 6 in which a bleaching solution was used as a first rinse water, the numbers of colonies in the first and second rinse drain waters were more than 100. In contrast, in the washings 7 and 8 in which a 50 ppm electrolyzed water was used as a first rinse water, the numbers of colonies in the first rinse drain water were 0 in both the washings 7 and 8 and the numbers of colonies in the second rinse drain water were 23 in the washing 7 and 4 in the washing 8. This showed that the 50 ppm electrolyzed water has better disinfectant properties than the 100 ppm bleaching solution. It was also found that, since the number of colonies in the second rinse drain water was small in the washing 7, a two-minute rinse is sufficient for disinfection when the amount of laundry is small.

TABLE 4

| | | First rinse | Second rinse | Specimen 1 | Specimen 2 |
|---|---|---|---|---|---|
| | | water | water | (number of | (number of |
| | Laundry | (rinse time) | (rinse time) | colonies) | colonies) |
| Washing 4 | Cultured towel | Tap water | Tap water | First rinse | Second rinse |
| (Comparative | (half) | (2 minutes) | (2 minutes) | drain water | drain water |
| Example) | | | | (800) | (198) |
| Washing 5 | Cultured towel | 100 ppm | Tap water | First rinse | Second rinse |
| (Comparative | (half) | bleaching | (2 minutes) | drain water | drain water |
| Example) | | solution | | (172) | (117) |
| | | (2 minutes) | | | |
| Washing 6 | Cultured towel | 100 ppm | Tap water | First rinse | Second rinse |
| (Comparative | (half) | bleaching | (2 minutes) | drain water | drain water |
| Example) | | solution | | (303) | (138) |
| | | (10 minutes) | | | |
| Washing 7 | Cultured towel | 50 ppm | Tap water | First rinse | Second rinse |
| (Example) | (half) | electrolyzed | (2 minutes) | drain water | drain water |
| | | water | | (0) | (23) |
| | | (2 minutes) | | | |
| Washing 8 | Cultured towel | 50 ppm | Tap water | First rinse | Second rinse |
| (Example) | (half) | electrolyzed | (2 minutes) | drain water | drain water |
| | | water | | (0) | (4) |
| | | (10 minutes) | | | |

### Washing Experiment 3

In the washing experiment 3, half of a towel in which general live bacteria were cultured with cow's milk was used as laundry. Washing was performed through the process illustrated in FIG. 12(a) while the effective chlorine concentration of the electrolyzed water used as a rinse water and the first rinse time were changed, and the microorganisms test was performed on the rinse drain water. Furthermore, a washing experiment of Comparative Example was performed in which tap water was used as a rinse water.

Table 5 shows the washing conditions of washings 9 to 17 and the determined numbers of colonies.

In the washings 10 to 13 in which a 50 ppm electrolyzed water was used as a first rinse water and the washings 14 to 17 in which a 20 ppm electrolyzed water was used as a first rinse water, the numbers of colonies in the first rinse drain water were 0 or 1 and the numbers of colonies in the second rinse drain water were 30 or less. This showed that the 20 ppm electrolyzed water has sufficiently good disinfectant properties. It was also found that the 20 ppm electrolyzed water has better disinfectant properties than the 100 ppm bleaching solution.

TABLE 5

| | | First rinse | Second rinse | Specimen 1 | Specimen 2 |
|---|---|---|---|---|---|
| | | water | water | (number of | (number of |
| | Laundry | (rinse time) | (rinse time) | colonies) | colonies) |
| Washing 9 | Cultured towel | Tap water | Tap water | First rinse | Second rinse |
| (Comparative | (half) | (2 minutes) | (2 minutes) | drain water | drain water |
| Example) | | | | (222) | (162) |
| Washing 10 | Cultured towel | 50 ppm | Tap water | First rinse | Second rinse |
| (Example) | (half) | electrolyzed | (2 minutes) | drain water | drain water |
| | | water | | (1) | (8) |
| | | (2 minutes) | | | |
| Washing 11 | Cultured towel | 50 ppm | Tap water | First rinse | Second rinse |
| (Example) | (half) | electrolyzed | (2 minutes) | drain water | drain water |
| | | water | | (0) | (12) |
| | | (10 minutes) | | | |
| Washing 12 | Cultured towel | 50 ppm | Tap water | First rinse | Second rinse |
| (Example) | (half) | electrolyzed | (2 minutes) | drain water | drain water |
| | | water | | (1) | (1) |
| | | (20 minutes) | | | |
| Washing 13 | Cultured towel | 50 ppm | Tap water | First rinse | Second rinse |
| (Example) | (half) | electrolyzed | (2 minutes) | drain water | drain water |
| | | water | | (0) | (0) |
| | | (30 minutes) | | | |
| Washing 14 | Cultured towel | 20 ppm | Tap water | First rinse | Second rinse |
| (Example) | (half) | electrolyzed | (2 minutes) | drain water | drain water |
| | | water | | (0) | (30) |
| | | (2 minutes) | | | |
| Washing 15 | Cultured towel | 20 ppm | Tap water | First rinse | Second rinse |
| (Example) | (half) | electrolyzed | (2 minutes) | drain water | drain water |
| | | water | | (0) | (8) |
| | | (10 minutes) | | | |
| Washing 16 | Cultured towel | 20 ppm | Tap water | First rinse | Second rinse |
| (Example) | (half) | electrolyzed | (2 minutes) | drain water | drain water |
| | | water | | (0) | (3) |
| | | (20 minutes) | | | |
| Washing 17 | Cultured towel | 20 ppm | Tap water | First rinse | Second rinse |
| (Example) | (half) | electrolyzed | (2 minutes) | drain water | drain water |
| | | water | | (0) | (6) |
| | | (30 minutes) | | | |

### Washing Experiment 4

In the washing experiment 4, half of a towel in which general live bacteria were cultured with cow's milk was used as laundry. Washing was performed through the process (triple rinsed) illustrated in FIG. 12(b) using a 20 ppm electrolyzed water or a 50 ppm electrolyzed water as a second rinse water while the second rinse time was changed, and the microorganisms test was performed on the rinse drain water. Furthermore, a washing experiment of Comparative Example was performed in which tap water was used as a rinse water.

Table 6 shows the washing conditions of washings 18 to 24 and the determined numbers of colonies.

In the washings 19 to 23 in which a 50 ppm electrolyzed water was used as a second rinse water, the numbers of colonies in the second and third rinse drain waters were 20 or less. In the washing 24 in which a 20 ppm electrolyzed water was used, the number of colonies in the third rinse water was also 40 or less. It was found from the washing experiment 4 that another rinse step before the rinse step with the electrolyzed water produces almost no effect. Therefore, it is believed that the washing cost can be further reduced when the first rinse step is performed using the electrolyzed water.

TABLE 6

| | | First rinse | Second rinse | Third rinse | Specimen 1 | Specimen 2 | Specimen 3 |
|---|---|---|---|---|---|---|---|
| | | water | water | water | (number of | (number of | (number of |
| | Laundry | (rinse time) | (rinse time) | (rinse time) | colonies) | colonies) | colonies) |
| Washing 18 | Cultured | Tap water | Tap water | Tap water | First rinse | Second rinse | Third rinse |
| (Comparative | towel (half) | (2 minutes) | (2 minutes) | (2 minutes) | drain water | drain water | drain water |
| Example) | | | | | (239) | (185) | (210) |
| Washing 19 | Cultured | Tap water | 50 ppm | Tap water | First rinse | Second rinse | Third rinse |
| (Example) | towel (half) | (2 minutes) | electrolyzed water | (2 minutes) | drain water | drain water | drain water |
| | | | (2 minutes) | | (273) | (0) | (17) |
| Washing 20 | Cultured | Tap water | 50 ppm | Tap water | First rinse | Second rinse | Third rinse |
| (Example) | towel (half) | (2 minutes) | electrolyzed water | (2 minutes) | drain water | drain water | drain water |
| | | | (5 minutes) | | (many) | (0) | (17) |
| Washing 21 | Cultured | Tap water | 50 ppm | Tap water | First rinse | Second rinse | Third rinse |
| (Example) | towel (half) | (2 minutes) | electrolyzed water | (2 minutes) | drain water | drain water | drain water |
| | | | (10 minutes) | | (many) | (0) | (4) |
| Washing 22 | Cultured | Tap water | 50 ppm | Tap water | First rinse | Second rinse | Third rinse |
| (Example) | towel (half) | (2 minutes) | electrolyzed water | (2 minutes) | drain water | drain water | drain water |
| | | | (20 minutes) | | (many) | (0) | (0) |
| Washing 23 | Cultured | Tap water | 50 ppm | Tap water | First rinse | Second rinse | Third rinse |
| (Example) | towel (half) | (2 minutes) | electrolyzed water | (2 minutes) | drain water | drain water | drain water |
| | | | (30 minutes) | | (240) | (0) | (2) |
| Washing 24 | Cultured | Tap water | 20 ppm | Tap water | First rinse | Second rinse | Third rinse |
| (Example) | towel (half) | (2 minutes) | electrolyzed water | (2 minutes) | drain water | drain water | drain water |
| | | | (2 minutes) | | (380) | (0) | (35) |

### Washing Experiment 5

In the washing experiment 5, half of a towel in which general live bacteria were cultured with cow's milk was used as laundry. Washing was performed through the process (double rinsed) illustrated in FIG. 12(a) using a 50 ppm electrolyzed water as a wash water and a first rinse water, and the microorganisms test was performed on the wash drain water and the rinse drain water. Furthermore, as

Comparative Examples, washing was performed using a 100 ppm bleaching solution as a wash water and the microorganisms test was performed.

The first to third rinse stages illustrated in FIG. 12 include a spin step, a rinse step, and a drain step. In each of the rinse stages, in reality, different rinses are sometimes performed at different timings. For example, after rinsing with stored water, the rinsing is stopped once and water is supplied, and then rinsing is performed while water is supplied. In this specification, they are included in a single rinse stage and are not differentiated. The single rinse stage is defined by complete draining, typically by spinning.

In the case where a wash tub and a spin tub are separated as in the case of twin-tub washing machines and a spin step requires some effort, the rinse stage is defined by complete draining normally performed between a wash step and a rinse step and between rinse steps in a twin-tub washing machine. The complete draining herein does not include draining of water that overflows during rinsing with water being supplied and draining in which water is intentionally left in a tub. Complete draining does not mean that water unintentionally left in a hollow of a tub or in laundry needs to be drained.

Table 7 shows the washing conditions of washings 25 to 28 and the determined numbers of colonies.

In the washings 25 and 26 in which a 50 ppm electrolyzed water and a 100 ppm bleaching solution were used as a wash water, respectively, the numbers of colonies in the wash drain water and the first and second rinse drain waters were more than 100, but the number of colonies in the rinse drain water was smaller in the washing 25 in which a 50 ppm electrolyzed water was used as a wash water. This showed that better disinfectant properties are also provided using a 50 ppm electrolyzed water than using a 100 ppm bleaching solution in the wash step. However, the number of colonies in the rinse drain water was smaller in the washings 27 and 28 in which a 50 ppm electrolyzed water was used as a first rinse water than in the washing 25. This showed that the electrolyzed water is desirably used as a rinse water.

The electrolyzed water may also be used in the wash step in addition to the rinse step. If any of tap water, a commercially available bleaching solution, and an electrolyzed water is used in the wash step, a commercially available chlorinated bleaching agent (sodium hypochlorite) or an electrolyzed water is preferably used. If disinfectant properties are given high priority, an electrolyzed water is most preferably used.

TABLE 7

| | | | First rinse | Second | Specimen 1 | Specimen 2 | Specimen 3 |
|---|---|---|---|---|---|---|---|
| | | Wash water | water | rinse water | (number of | (number of | (number of |
| | Laundry | (washing time) | (rinse time) | (rinse time) | colonies) | colonies) | colonies) |
| Washing 25 | Cultured | 50 ppm | Tap water | Tap water | Wash drain | First rinse | Second |
| (Example) | towel (half) | electrolyzed | (2 minutes) | (2 minutes) | water | drain water | rinse drain |
| | | water | | | (400) | (179) | water |
| | | (4 minutes) | | | | | (150) |
| Washing 26 | Cultured | 100 ppm | Tap water | Tap water | Wash drain | First rinse | Second |
| (Comparative | towel (half) | bleaching | (2 minutes) | (2 minutes) | water | drain water | rinse drain |
| Example) | | solution | | | (440) | (420) | water |
| | | (4 minutes) | | | | | (400) |
| Washing 27 | Cultured | 50 ppm | 50 ppm | Tap water | Wash drain | First rinse | Second |
| (Example) | towel (half) | electrolyzed | electrolyzed | (2 minutes) | water | drain water | rinse drain |
| | | water | water | | (420) | (0) | water |
| | | (4 minutes) | (2 minutes) | | | | (11) |
| Washing 28 | Cultured | Tap water | 50 ppm | Tap water | Wash drain | First rinse | Second |
| (Example) | towel (half) | (4 minutes) | electrolyzed | (2 minutes) | water | drain water | rinse drain |
| | | | water | | (many) | (0) | water |
| | | | (2 minutes) | | | | (21) |

The spin step between the wash step and the rinse step that uses the electrolyzed water is preferably performed in the same manner as that of typical washing or more thoroughly. When laundry with many bacteria or heavily soiled laundry such as a dustcloth is washed, disinfection cannot be completely achieved only in the wash step even when a detergent or a bleaching agent is used under typical conditions, and many bacteria are also contained in the wash water after the wash step. Therefore, if spinning is insufficiently performed, a water component of the wash water and bacteria contained in the wash water are left in the laundry and the like. Consequently, a disinfectant component of the electrolyzed water is excessively consumed during rinsing with the electrolyzed water, and the essential disinfection of the laundry may be insufficiently performed. When spinning is thoroughly performed, insufficient disinfection can be suppressed.

If water is left in the laundry after the spin step before the rinse step and, in particular, there are many minute gaps like fibers, the electrolyzed water does not readily penetrate into the laundry, and bacteria that adhere in the depths of fibers of the cloth may be not completely removed. Therefore, spinning before the electrolyzed water is supplied is preferably performed in a typical manner or more thoroughly. Spinning is more thoroughly performed by, for example, lengthening the spinning time, increasing the rotational speed, or employing a combined method thereof. Alternatively, removal of water may be facilitated by air blowing or heating.

When a NaCl component is contained in the electrolyzed water, the penetration into details of laundry is facilitated and thus disinfection is facilitated unless the laundry is extremely hydrophobic or the pH of the electrolyzed water is low. Even when the laundry is hydrophobic or the pH of the electrolyzed water is low, no adverse effect such as degradation of disinfectant properties is exerted. Therefore, the electrolyzed water preferably contains a NaCl component. In particular, when an electrolyzed water having a pH of 6.5 or more contains NaCl, the effect is produced on hydrophilic laundry such as clothes. The electrolyzed water preferably has a pH of 7.0 or more because the effect is further produced.

### Reference Signs List

1 electrolysis electrode pair

3 anode

4 cathode

5 electrolysis unit

7 electrolytic solution tank

8 pump

10 electrolytic solution supplying unit

12 electrolytic stock solution

13 electrolyte for generating electrolyzed water

14 concentrated electrolytic solution

15 flow-out port

18 electrolyzed water

19 stirring unit

20 electrolyzed water diluting unit

22 solenoid-controlled valve

24 electrolytic solution diluting unit

25 electrolytic solution preparing unit

30 electrolyzed water generating device

32 flow inlet (stirring unit)

33 flow outlet (stirring unit)

35 air bubble dividing unit

37 barrier

40 flux direction of electrolyzed water that flows in through flow inlet

42 flux direction of electrolyzed water that moves toward flow outlet

45 air bubbles

1. An electrolyzed water generating device comprising an electrolytic solution supplying unit and an electrolysis unit including an electrolysis electrode pair,
wherein the electrolytic solution supplying unit is provided so as to supply an aqueous solution of an electrolyte for generating electrolyzed water to the electrolysis unit,
the electrolysis unit is provided so that the aqueous solution of the electrolyte for generating electrolyzed water is electrolyzed using the electrolysis electrode pair to generate an electrolyzed water,
the electrolyte for generating electrolyzed water contains an alkali metal chloride and a substance that makes an aqueous solution acidic, and
the electrolyzed water generating device generates an electrolyzed water having a pH of more than 6.5 and less than 8.0.

2. The electrolyzed water generating device according to claim 1, wherein an electrolyzed water having an effective chlorine concentration of 10 ppm or more and 100 ppm or less is generated.

3. The electrolyzed water generating device according to claim 1, wherein the alkali metal chloride is at least one of sodium chloride and potassium chloride.

4. The electrolyzed water generating device according to claim 1, wherein the substance that makes an aqueous solution acidic is hydrogen chloride.

5. An electrolyzed water generating device comprising an electrolytic solution supplying unit, an electrolysis unit including an electrolysis electrode pair, and a diluting unit,
wherein the electrolytic solution supplying unit is provided so as to supply an aqueous solution of an electrolyte for generating electrolyzed water to the electrolysis unit through at least a portion below the middle of the electrolysis unit,
the electrolysis unit is provided so that the aqueous solution of the electrolyte for generating electrolyzed water is electrolyzed using the electrolysis electrode pair to generate an electrolyzed water, and the generated electrolyzed water flows out through at least a portion above the middle of the electrolysis unit,
the diluting unit is provided so as to mix the electrolyzed water that has flowed out from the electrolysis unit with water, and
the diluting unit is provided so that a flow of the electrolyzed water joins a flow of water flowing in a substantially horizontal direction, or at least a pipe which extends in a horizontal direction and through which the electrolyzed water diluted by the diluting unit flows is longer than a pipe which extends in a vertical direction and through which the electrolyzed water diluted by the diluting unit flows.

6. An electrolyzed water generating device comprising an electrolytic solution supplying unit, an electrolysis unit including an electrolysis electrode pair, and a stirring unit,
wherein the electrolytic solution supplying unit is provided so as to supply an aqueous solution of an electrolyte for generating electrolyzed water to the electrolysis unit,
the electrolysis unit is provided so that the aqueous solution of the electrolyte for generating electrolyzed water is electrolyzed using the electrolysis electrode pair to generate an electrolyzed water,
the stirring unit includes a flow inlet through which the electrolyzed water generated by the electrolysis unit flows in and a flow outlet through which the electrolyzed water flows out from the stirring unit,
the flow outlet is provided in an upper portion of the stirring unit to prevent gas from being easily accumulated,
the flow inlet is provided below the flow outlet, and
the flow inlet and the flow outlet have a relationship in which a flux direction of an electrolyzed water that flows in through the flow inlet is not parallel to a flux direction of an electrolyzed water that flows toward the flow outlet, a relationship in which, when projected in a vertical direction, these flux directions do not overlap each other, or a relationship in which an obstacle is present on a line segment that connects the flow inlet and the flow outlet.

7. An electrolyte for generating electrolyzed water, the electrolyte comprising an alkali metal chloride and a substance that makes an aqueous solution acidic.

8. The electrolyte for generating electrolyzed water according to claim 7, the electrolyte comprising water serving as a solvent,
wherein the substance that makes an aqueous solution acidic is hydrogen chloride, and
a total concentration of the alkali metal chloride and the hydrogen chloride is 1% or more and 23% or less, and a ratio of hydrogen chloride/alkali metal chloride is 1/20 or more and ½ or less.

9. An electrolyzed water for disinfection, generated by electrolyzing an aqueous solution of an electrolyte for generating electrolyzed water, the electrolyte containing an alkali metal chloride and a substance that makes an aqueous solution acidic,
wherein the electrolyzed water for disinfection has a pH of more than 6.5 and less than 8.0.

10. The electrolyzed water for disinfection according to claim 9, comprising an alkali metal chloride, hypochlorous acid, and a hypochlorite,
wherein a concentration decreases in the order of the alkali metal chloride, the hypochlorous acid, and the hypochlorite.

11. A washer comprising the electrolyzed water generating device according to claim 1,
wherein the washer is provided so as to perform a wash step of removing soils, a disinfecting and cleaning step of performing disinfection and cleaning using the electrolyzed water generated by the electrolyzed water generating device, and a rinse step, and
the disinfecting and cleaning step is performed between a spin step after the wash step and the rinse step performed after water used in the disinfecting and cleaning step is drained and removed.
